(19) United States
(12) Patent Application Publication
(10) Pub. No.: US 2008/0161269 A1
(43) Pub. Date: Jul. 3, 2008

(54) Compounds 620

(75) Inventors: Stefan Berg, Sodertalje (SE); Sofia Karlstrom, Sodertalje (SE); Karin Kolmodin, Sodertalje (SE); Johan Lindstrom, Sodertalje (SE); Jan-Erik Nystrom, Sodertalje (SE); Fernando Sehgelmeble, Sodertalje (SE); Peter Soderman, Sodertalje (SE)

Correspondence Address: PEPPER HAMILTON LLP, 400 BERWYN PARK, 899 CASSATT ROAD, BERWYN, PA, 19312-1183, US

(73) Assignees: ASTRAZENECA AB, Sodertalje (SE); ASTEX THERAPEUTICS LIMITED, Cambridge (GB)

(21) Appl. No.: 11/959,604

(22) Filed: Dec. 19, 2007

Related U.S. Application Data
(60) Provisional application No. 60/870,953, filed on Dec. 20, 2006.

(51) Int. Cl. A61K 31/695; C07F 7/08; A61P 25/00

(52) U.S. Cl. 514/63; 548/110; 546/14; 544/229

(57) ABSTRACT

This invention relates to novel compounds having the structural formula I below:

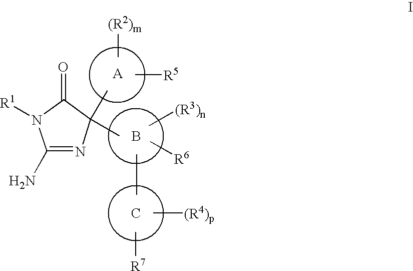

and to their pharmaceutically acceptable salt, compositions and methods of use. These novel compounds provide a treatment or prophylaxis of cognitive impairment, Alzheimer Disease, neurodegeneration and dementia.

The present invention relates to novel compounds and their pharmaceutical compositions. In addition, the present invention relates to therapeutic methods for the treatment and/or prevention of Aβ-related pathologies such as Downs syndrome, β-amyloid angiopathy such as but not limited to cerebral amyloid angiopathy or hereditary cerebral hemorrhage, disorders associated with cognitive impairment such as but not limited to MCI (“mild cognitive impairment”), Alzheimer Disease, memory loss, attention deficit symptoms associated with Alzheimer disease, neurodegeneration associated with diseases such as Alzheimer disease or dementia including dementia of mixed vascular and degenerative origin, pre-senile dementia, senile dementia and dementia associated with Parkinson's disease, progressive supranuclear palsy or cortical basal degeneration.

### BACKGROUND OF THE INVENTION

Several groups have identified and isolated aspartate proteinases that have β-secretase activity (Hussain et al., 1999; Lin et. al, 2000; Yan et. al, 1999; Sinha et. al., 1999 and Vassar et. al., 1999). β-secretase is also known in the literature as Asp2 (Yan et. al, 1999), Beta site APP Cleaving Enzyme (BACE) (Vassar et. al., 1999) or memapsin-2 (Lin et al., 2000). BACE was identified using a number of experimental approaches such as EST database analysis (Hussain et al. 1999); expression cloning (Vassar et al. 1999); identification of human homologs from public databases of predicted C. elegans proteins (Yan et al. 1999) and finally utilizing an inhibitor to purify the protein from human brain (Sinha et al. 1999). Thus, five groups employing three different experimental approaches led to the identification of the same enzyme, making a strong case that BACE is a β-secretase. Mention is also made of the patent literature: WO96/40885, EP871720, U.S. Pat. Nos. 5,942,400 and 5,744,346, EP855444, U.S. Pat. No. 6,319,689, WO99/64587, WO99/31236, EP1037977, WO00/17369, WO01/23533, WO0047618, WO00/58479, WO00/69262, WO01/00663, WO01/00665, U.S. Pat. No. 6,313,268.

BACE was found to be a pepsin-like aspartic proteinase, the mature enzyme consisting of the N-terminal catalytic domain, a transmembrane domain, and a small cytoplasmic domain. BACE has an optimum activity at pH 4.0-5.0 (Vassar et al, 1999) and is inhibited weakly by standard pepsin inhibitors such as pepstatin. It has been shown that the catalytic domain minus the transmembrane and cytoplasmic domain has activity against substrate peptides (Lin et al, 2000). BACE is a membrane bound type 1 protein that is synthesized as a partially active proenzyme, and is abundantly expressed in brain tissue. It is thought to represent the major β-secretase activity, and is considered to be the rate-limiting step in the production of amyloid-β-protein (Aβ). It is thus of special interest in the pathology of Alzheimer's disease, and in the development of drugs as a treatment for Alzheimer's disease.

Aβ or amyloid-β-protein is the major constituent of the brain plaques which are characteristic of Alzheimer's disease (De Strooper et al, 1999). Aβ is a 39-42 residue peptide formed by the specific cleavage of a class 1 transmembrane protein called APP, or amyloid precursor protein. Cleavage of APP by BACE generates the extracellular soluble APP□ fragment and the membrane bound CTF□ (C99) fragment that is subsequently cleaved by □-secretase to generate A□ peptide.

Alzheimer's disease (AD) is estimated to afflict more than 20 million people worldwide and is believed to be the most common form of dementia. Alzheimer's disease is a progressive dementia in which massive deposits of aggregated protein breakdown products—amyloid plaques and neurofibrillary tangles accumulate in the brain. The amyloid plaques are thought to be responsible for the mental decline seen in Alzheimer's patients.

The likelihood of developing Alzheimer's disease increases with age, and as the aging population of the developed world increases, this disease becomes a greater and greater problem. In addition to this, there is a familial link to Alzheimer's disease and consequently any individuals possessing the double mutation of APP known as the Swedish mutation (in which the mutated APP forms a considerably improved substrate for BACE) have a much higher risk of developing AD, and also of developing the disease at an early age (see also U.S. Pat. No. 6,245,964 and U.S. Pat. No. 5,877,399 pertaining to transgenic rodents comprising APP-Swedish). Consequently, there is also a strong need for developing a compound that can be used in a prophylactic fashion for these individuals.

The gene encoding APP is found on chromosome 21, which is also the chromosome found as an extra copy in Down's syndrome. Down's syndrome patients tend to develop Alzheimer's disease at an early age, with almost all those over 40 years of age showing Alzheimer's-type pathology (Oyama et al., 1994). This is thought to be due to the extra copy of the APP gene found in these patients, which leads to overexpression of APP and therefore to increased levels of Aβ causing the high prevalence of Alzheimer's disease seen in this population. Thus, inhibitors of BACE could be useful in reducing Alzheimer's-type pathology in Down's syndrome patients.

Drugs that reduce or block BACE activity should therefore reduce Aβ levels and levels of fragments of Aβ in the brain, or elsewhere where Aβ or fragments thereof deposit, and thus slow the formation of amyloid plaques and the progression of AD or other maladies involving deposition of Aβ or fragments thereof (Yankner, 1996; De Strooper and Konig, 1999). BACE is therefore an important candidate for the development of drugs as a treatment and/or prophylaxis of Aβ-related pathologies such as Downs syndrome, β-amyloid angiopathy such as but not limited to cerebral amyloid angiopathy or hereditary cerebral hemorrhage, disorders associated with cognitive impairment such as but not limited to MCI (“mild cognitive impairment”), Alzheimer Disease, memory loss, attention deficit symptoms associated with Alzheimer disease, neurodegeneration associated with diseases such as Alzheimer disease or dementia including dementia of mixed vascular and degenerative origin, pre-senile dementia, senile dementia and dementia associated with Parkinson's disease, progressive supranuclear palsy or cortical basal degeneration.

It would therefore be useful to inhibit the deposition of Aβ and portions thereof by inhibiting BACE through inhibitors such as the compounds provided herein.

The therapeutic potential of inhibiting the deposition of Aβ has motivated many groups to isolate and characterize secretase enzymes and to identify their potential inhibitors (see, e.g., WO01/23533 A2, EP0855444, WO00/17369, WO00/58479, WO00/47618, WO00/77030, WO01/00665, WO01/00663, WO01/29563, WO02/25276, U.S. Pat. No. 5,942,400, U.S. Pat. No. 6,245,884, U.S. Pat. No. 6,221,667, U.S. Pat. No. 6,211,235, WO02/02505, WO02/02506, WO02/02512, WO02/02518, WO02/02520, WO02/14264, WO05/058311, WO05/097767, WO06/041404, WO06/041405, WO06/0065204, WO06/0065277, US2006287294, WO06/138265, US20050282826, US20050282825, US20060281729, WO06/138217, WO06/138230, WO06/138264, WO06/138265, WO06/138266, WO06/099379, WO06/076284, US20070004786, US20070004730, WO07/011833, WO07/011810, US20070099875, US20070099898, WO07/058,601, WO07/058,581, WO07/058,580, WO07/058,583, WO07/058,582, WO07/058,602, WO07/073,284, WO07/049,532, WO07/038,271, WO07/016,012, WO07/005,366, WO07/005,404, WO06/0009653).

### DISCLOSURE OF THE INVENTION

In one aspect of the invention there is provided novel BACE inhibitors of formula I:

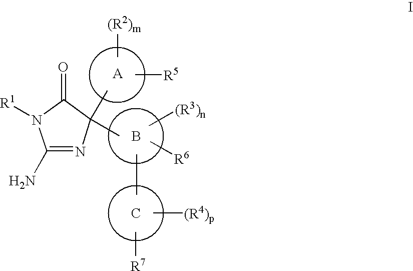

wherein
A is selected from C₁₋₆alkyl, C₂₋₆alkenyl, C₂₋₆alkynyl, C₃₋₆cycloalkyl, C₅₋₇cycloalkenyl, C₆₋₈cycloalkynyl, aryl, heteroaryl, heterocyclyl, C₁₋₆alkylC₃₋₆cycloalkyl, C₁₋₆alkylaryl, C₁₋₆alkylheteroaryl and C₁₋₆alkylheterocyclyl, wherein said C₁₋₆alkyl, C₂₋₆alkenyl, C₂₋₆alkynyl, C₃₋₆cycloalkyl, C₅₋₇cycloalkenyl, C₆₋₈cycloalkynyl, aryl, heteroaryl, heterocyclyl, C₁₋₆alkylC₃₋₆cycloalkyl, C₁₋₆alkylaryl, C₁₋₆alkylheteroaryl or C₁₋₆alkylheterocyclyl is optionally substituted with one or more R⁵;
B is selected from aryl and heteroaryl, wherein said aryl or heteroaryl is optionally substituted with one or more R⁶;
C is selected from aryl, heterocyclyl and heteroaryl, wherein said aryl, heterocyclyl or heteroaryl is optionally substituted with one or more R⁷;
R¹ is selected from hydrogen, C₁₋₆alkyl, C₂₋₆alkenyl, C₂₋₆alkynyl, C₃₋₆cycloalkyl, C₅₋₇cycloalkenyl, C₆₋₈cycloalkynyl, aryl, heteroaryl, heterocyclyl, C₁₋₆alkylC₃₋₆cycloalkyl, C₁₋₆alkylaryl, C₁₋₆alkylheteroaryl and C₁₋₆alkylheterocyclyl, wherein said C₁₋₆alkyl, C₂₋₆alkenyl, C₂₋₆alkynyl, C₃₋₆cycloalkyl, C₅₋₇cycloalkenyl, C₆₋₈cycloalkynyl, aryl, heteroaryl, heterocyclyl, C₁₋₆alkylC₃₋₆cycloalkyl, C₁₋₆alkylaryl, C₁₋₆alkylheteroaryl or C₁₋₆alkylheterocyclyl is optionally substituted with one, two or three D;

### R², R³ and R⁴ is Si(R⁸)₃;

R⁵, R⁶ and R⁷ is independently selected from halogen, nitro, CHO, C₀₋₆alkylCN, OC₁₋₆alkylCN, C₀₋₆alkylOR⁹, OC₂₋₆alkylOR⁹, C₀₋₆alkylNR⁹R¹⁰, OC₂₋₆alkylNR⁹R¹⁰, OC₂₋₆alkylOC₂₋₆alkylNR⁹R¹⁰, NR⁹OR¹⁰, C₀₋₆alkylCO₂R⁹, OC₁₋₆alkylCO₂R⁹, C₀₋₆alkylCONR⁹R¹⁰, OC₁₋₆alkylCONR⁹R¹⁰, OC₂₋₆alkylNR⁹(CO)R¹⁰, C₀₋₆alkylNR⁹(CO)R¹⁰, O(CO)NR⁹R¹⁰, NR⁹(CO)OR¹⁰, NR⁹(CO)NR⁹R¹⁰, O(CO)OR⁹, O(CO)R⁹, C₀₋₆alkylCOR⁹, OC₁₋₆alkylCOR⁹, NR⁹(CO)(CO)R⁹, NR⁹(CO)(CO)NR⁹R¹⁰, C₀₋₆alkylSR⁹, C₀₋₆alkyl(SO₂)NR⁹R¹⁰, OC₁₋₆alkylNR⁹(SO₂)R¹⁰, OC₀₋₆alkyl(SO₂)NR⁹R¹⁰, C₀₋₆alkyl(SO)NR⁹R¹⁰, OC₁₋₆alkyl(SO)NR⁹R¹⁰, OSO₂R⁹, SO₃R⁹, C₀₋₆alkylNR⁹(SO₂)NR⁹R¹⁰, C₀₋₆alkylNR⁹(SO)R¹⁰, OC₂₋₆alkylNR⁹(SO)R⁹, OC₁₋₆alkylSO₂R⁹, C₁₋₆alkylSO₂R⁹, C₀₋₆alkylSOR⁹, C₁₋₆alkyl, C₂₋₆alkenyl, C₂₋₆alkynyl, C₀₋₆alkylC₃₋₆cycloalkyl, C₀₋₆alkylC₅₋₇cycloalkenyl, C₀₋₆alkylC₆₋₈cycloalkynyl, C₀₋₆alkylaryl, C₀₋₆alkylheteroaryl, C₀₋₆alkylheterocyclyl, and OC₂₋₆alkylheterocyclyl, wherein said C₁₋₆alkyl, C₂₋₆alkenyl, C₂₋₆alkynyl, C₀₋₆alkylC₃₋₆cycloalkyl, C₀₋₆alkylC₅₋₇cycloalkenyl, C₀₋₆alkylC₆₋₈cycloalkynyl, C₀₋₆alkylaryl, C₀₋₆alkylheteroaryl, C₀₋₆alkylheterocyclyl or OC₂₋₆alkylheterocyclyl is optionally substituted by one or more D, and wherein the individual aryl or heteroaryl groups of C₀₋₆alkylaryl or C₀₋₆alkylheteroaryl is optionally fused with a 4, 5, 6 or 7 membered cycloalkyl, cycloalkenyl or heterocyclyl group to form a bicyclic ring system where the bicyclic ring system is optionally substituted with from one or more D;
R⁸ is selected from hydrogen, C₁₋₆alkyl, C₂₋₆alkenyl, C₂₋₆alkynyl, C₀₋₆alkylOR¹¹, C₀₋₆alkylC₃₋₆cycloalkyl, C₀₋₆alkylC₅₋₇cycloalkenyl, C₀₋₆alkylC₆₋₈cycloalkynyl, C₀₋₆alkylaryl, C₀₋₆alkylheteroaryl, C₀₋₆alkylheterocyclyl and C₀₋₆alkylNR¹¹R¹², wherein said C₁₋₆alkyl, C₂₋₆alkenyl, C₂₋₆alkynyl, C₀₋₆alkylC₃₋₆cycloalkyl, C₀₋₆alkylC₅₋₇cycloalkenyl, C₀₋₆alkylC₆₋₈cycloalkynyl, C₀₋₆alkylaryl, C₀₋₆alkylheteroaryl or C₀₋₆alkylheterocyclyl is optionally substituted with one or more D;
R⁹ and R¹⁰ are independently selected from hydrogen, halogen, C₁₋₆alkyl, C₂₋₆alkenyl, C₂₋₆alkynyl, C₀₋₆alkylC₃₋₆cycloalkyl, C₀₋₆alkylC₅₋₇cycloalkenyl, C₀₋₆alkylC₆₋₈cycloalkynyl, C₂₋₆alkenylC₃₋₆cycloalkyl, C₂₋₆alkenylC₅₋₇cycloalkenyl, C₂₋₆alkenylC₆₋₈cycloalkynyl, C₀₋₆alkylaryl, C₀₋₆alkylheteroaryl, C₀₋₆alkylheterocyclyl, C₀₋₆alkylOR¹¹, C₀₋₆alkyNR¹¹R¹² aryl, and heteroaryl, wherein said C₁₋₆alkyl, C₂₋₆alkenyl, C₂₋₆alkynyl, C₀₋₆alkylC₃₋₆cycloalkyl, C₀₋₆alkylC₅₋₇cycloalkenyl, C₀₋₆alkylC₆₋₈cycloalkynyl, C₀₋₆alkylaryl, C₀₋₆alkylheteroaryl, C₀₋₆alkylheterocyclyl, aryl or heteroaryl is optionally substituted by one or more D; or
R⁹ and R¹⁰ may together form a 4 to 6 membered heterocyclic ring containing one or more heteroatoms selected from N, O or S that is optionally substituted with one or more D;
whenever two R⁹ groups occur in the structure they may optionally together form a 5 or 6 membered heterocyclic ring containing one or more heteroatoms selected from N, O or S, that is optionally substituted with one or more D;

R¹¹ and R¹² are independently selected from hydrogen, halogen, C₁₋₆alkyl, C₂₋₆alkenyl, C₂₋₆alkynyl, C₀₋₆alkylC₃₋₆cycloalkyl, C₀₋₆alkylC₅₋₇cycloalkenyl, C₀₋₆alkylC₆₋₈cycloalkynyl, C₀₋₆alkylaryl, C₀₋₆alkylheterocyclyl and C₀₋₆alkylheteroaryl, wherein said C₁₋₆alkyl, C₂₋₆alkenyl, C₂₋₆alkynyl, C₀₋₆alkylC₃₋₆cycloalkyl, C₀₋₆alkylC₅₋₇cycloalkenyl, C₀₋₆alkylC₆₋₈cycloalkynyl, C₀₋₆alkylaryl, C₀₋₆alkylheteroaryl or C₀₋₆alkylheterocyclyl is optionally substituted with one or more D; or

R¹¹ and R¹² may together form a 4 to 6 membered heterocyclic ring containing one or more heteroatoms selected from N, O or S optionally substituted with one or more D;
D is selected from halogen, nitro, COOH, CN, OR¹³, C₁₋₆alkyl, C₂₋₆alkenyl, C₂₋₆alkynyl, C₀₋₆alkylaryl, C₀₋₆alkylheteroaryl, C₀₋₆alkylC₃₋₆cycloalkyl, C₀₋₆alkylC₅₋₇cycloalkenyl, C₀₋₆alkylC₆₋₈cycloalkynyl, C₀₋₆alkylheterocyclyl, OC₂₋₆alkylNR¹³R¹⁴, NR¹³R¹⁴, CONR¹³R¹⁴NR¹³(CO)R¹⁴, O(CO)R¹³, (CO)O R¹³, COR¹³ (SO₂)NR¹³R¹⁴, NSO₂R¹³, SO₂R¹³, SOR¹³ (CO)C₁₋₆alkylNR¹³R¹⁴, (SO₂)C₁₋₆alkylNR¹³R¹⁴, OSO₂R¹³ and SO₃R¹³, wherein said C₁₋₆alkyl, C₂₋₆alkenyl, C₂₋₆alkynyl, C₀₋₆alkylaryl, C₀₋₆alkylheteroaryl, C₀₋₆alkylheterocyclyl, C₀₋₆alkylC₃₋₆cycloalkyl C₀₋₆alkylC₅₋₇cycloalkenyl or C₀₋₆alkylC₆₋₈cycloalkynyl is optionally substituted with halogen, OSO₂R¹³, SO₃R¹³, nitro, CN, OR¹³, C₁₋₆alkyl;
R¹³ and R¹⁴ are independently selected from hydrogen, halogen, C₁₋₆alkyl, C₃₋₆cycloalkyl, aryl, heteroaryl or heterocyclyl wherein said C₁₋₆alkyl, C₃₋₆cycloalkyl, aryl, heteroaryl or heterocyclyl is optionally substituted with one, two or three hydroxy, CN, halo or C₁₋₃alkyloxy; or
R¹³ and R¹⁴ may together form a 4 to 6 membered heterocyclic ring containing one or more heteroatoms selected from N, O or S optionally substituted with hydroxy, C₁₋₃alkyloxy, cyano or halo;
m=0, 1, 2 or 3;
n=0, 1, 2 or 3;
p=0, 1, 2 or 3;
wherein one of m, n or p is at least 1;
as a free base or a pharmaceutically acceptable salt, solvate or solvate of a salt thereof.

The present invention further provides pharmaceutical compositions comprising as active ingredient a therapeutically effective amount of a compound of formula I in association with pharmaceutically acceptable excipients, carriers or diluents.

The present invention further provides methods of modulating activity of BACE comprising contacting the BACE enzyme with a compound of formula I.

The present invention further provides methods of treating or preventing an Aβ-related pathology in a patient, comprising administering to the patient a therapeutically effective amount of a compound of formula I.

The present invention further provides a compound described herein for use as a medicament.

In another aspect of the invention, there is provided a compound of formula I, wherein

A is selected from hydrogen, C₁₋₆alkyl, C₂₋₆alkenyl, C₂₋₆alkynyl, C₃₋₆cycloalkyl, C₅₋₇cycloalkenyl, C₆₋₈cycloalkynyl, aryl, heteroaryl, heterocyclyl, C₁₋₆alkylC₃₋₆cycloalkyl, C₁₋₆alkylaryl, C₁₋₆alkylheteroaryl and C₁₋₆alkylheterocyclyl, wherein said C₁₋₆alkyl, C₂₋₆alkenyl, C₂₋₆alkynyl, C₃₋₆cycloalkyl, C₅₋₇cycloalkenyl, C₆₋₈cycloalkynyl, aryl, heteroaryl, heterocyclyl, C₁₋₆alkylC₃₋₆cycloalkyl, C₁₋₆alkylaryl, C₁₋₆alkylheteroaryl or C₁₋₆alkylheterocyclyl is optionally substituted with one or more R⁵
B is selected from aryl and heteroaryl, wherein said aryl or heteroaryl is optionally substituted with one or more R⁶;
C is selected from hydrogen, aryl and heteroaryl, wherein said aryl or heteroaryl is optionally substituted with one or more R⁷;
R¹ is selected from hydrogen, C₁₋₆alkyl, C₂₋₆alkenyl, C₂₋₆alkynyl, C₃₋₆cycloalkyl, C₅₋₇cycloalkenyl, C₆₋₈cycloalkynyl, aryl, heteroaryl, heterocyclyl, C₁₋₆alkylC₃₋₆cycloalkyl, C₁₋₆alkylaryl, C₁₋₆alkylheteroaryl and C₁₋₆alkylheterocyclyl, wherein said C₁₋₆alkyl, C₂₋₆alkenyl, C₂₋₆alkynyl, C₃₋₆cycloalkyl, C₅₋₇cycloalkenyl, C₆₋₈cycloalkynyl, aryl, heteroaryl, heterocyclyl, C₁₋₆alkylC₃₋₆cycloalkyl, C₁₋₆alkylaryl, C₁₋₆alkylheteroaryl or C₁₋₆alkylheterocyclyl is optionally substituted with one, two or three D;

### R², R³ and R⁴ is Si(R⁸)₃;

R⁵, R⁶ or R⁷ is independently selected from halogen, nitro, CHO, C₀₋₆alkylCN, OC₁₋₆alkylCN, C₀₋₆alkylOR⁹, OC₂₋₆alkylOR⁹, C₀₋₆alkylNR⁹R¹⁰, OC₂₋₆alkylNR⁹R¹⁰, OC₂₋₆alkylOC₂₋₆alkylNR⁹R¹⁰, NR⁹OR¹⁰, C₀₋₆alkylCO₂R⁹, OC₁₋₆alkylCO₂R⁹, C₀₋₆alkylCONR⁹R¹⁰, OC₁₋₆alkylCONR⁹R¹⁰, OC₂₋₆alkylNR⁹(CO)R¹⁰, C₀₋₆alkylNR⁹(CO)R¹⁰, O(CO)NR⁹R¹⁰, NR⁹(CO)OR¹⁰, NR⁹(CO)NR⁹R¹⁰, O(CO)OR⁹, O(CO)R⁹, C₀₋₆alkylCOR⁹, OC₁₋₆alkylCOR⁹, NR⁹(CO)(CO)R⁹, NR⁹(CO)(CO)NR⁹R¹⁰, C₀₋₆alkylSR⁹, C₀₋₆alkyl(SO₂)NR⁹R¹⁰, OC₁₋₆alkylNR⁹(SO₂)R¹⁰, OC₀₋₆alkyl(SO₂)NR⁹R¹⁰, C₀₋₆alkyl(SO)NR⁹R¹⁰, OC₁₋₆alkyl(SO)NR⁹R¹⁰, OSO₂R⁹, SO₃R⁹, C₀₋₆alkylNR⁹(SO₂)NR⁹R¹⁰, C₀₋₆alkylNR⁹(SO)R¹⁰, OC₂₋₆alkylNR⁹(SO)R⁹, OC₁₋₆alkylSO₂R⁹, C₁₋₆alkylSO₂R⁹, C₀₋₆alkylSOR⁹, C₁₋₆alkyl, C₂₋₆alkenyl, C₂₋₆alkynyl, C₀₋₆alkylC₃₋₆cycloalkyl, C₀₋₆alkylC₅₋₇cycloalkenyl, C₀₋₆alkylC₆₋₈cycloalkynyl, C₀₋₆alkylaryl, C₀₋₆alkylheteroaryl, C₀₋₆alkylheterocyclyl, and OC₂₋₆alkylheterocyclyl, wherein any C₁₋₆alkyl, C₂₋₆alkenyl, C₂₋₆alkynyl, C₀₋₆alkylC₃₋₆cycloalkyl, C₀₋₆alkylC₅₋₇cycloalkenyl, C₀₋₆alkylC₆₋₈cycloalkynyl, C₀₋₆alkylaryl, C₀₋₆alkylheteroaryl, C₀₋₆alkylheterocyclyl or OC₂₋₆alkylheterocyclyl is optionally substituted by one or more D, and wherein the individual aryl or heteroaryl groups may be optionally fused with a 4, 5, 6 or 7 membered cycloalkyl, cycloalkenyl or heterocyclyl group to form a bicyclic ring system where the bicyclic ring system is optionally substituted with between one and four D;
R⁸ is selected from hydrogen, C₁₋₆alkyl, C₂₋₆alkenyl, C₂₋₆alkynyl, C₀₋₆alkylOR¹¹, C₀₋₆alkylC₃₋₆cycloalkyl, C₀₋₆alkylC₅₋₇cycloalkenyl, C₀₋₆alkylC₆₋₈cycloalkynyl, C₀₋₆alkylaryl, C₀₋₆alkylheteroaryl, C₀₋₆alkylheterocyclyl and C₀₋₆alkylNR¹¹R¹², wherein said C₁₋₆alkyl, C₂₋₆alkenyl, C₂₋₆alkynyl, C₀₋₆alkylC₃₋₆cycloalkyl, C₀₋₆alkylC₅₋₇cycloalkenyl, C₀₋₆alkylC₆₋₈cycloalkynyl, C₀₋₆alkylaryl, C₀₋₆alkylheteroaryl or C₀₋₆alkylheterocyclyl is optionally substituted by one or more D;
R⁹ and R¹⁰ are independently selected from hydrogen, halogen, C₁₋₆alkyl, C₂₋₆alkenyl, C₂₋₆alkynyl, C₀₋₆alkylC₃₋₆cycloalkyl, C₀₋₆alkylC₅₋₇cycloalkenyl, C₀₋₆alkylC₆₋₈cycloalkynyl, C₀₋₆alkylaryl, C₀₋₆alkylheteroaryl, C₀₋₆alkylheterocyclyl, C₀₋₆alkylOR¹¹ and C₀₋₆alkylNR¹¹R¹², wherein said C₁₋₆alkyl, C₂₋₆alkenyl, C₂₋₆alkynyl, C₀₋₆alkylC₃₋₆cycloalkyl, C₀₋₆alkylC₅₋₇cycloalkenyl, C₀₋₆alkylC₆₋₈cycloalkynyl, C₀₋₆alkylaryl, C₀₋₆alkylheteroaryl or C₀₋₆alkylheterocyclyl is optionally substituted by one or more D; or
R⁹ and R¹⁰ may together form a 4 to 6 membered heterocyclic ring containing one or more heteroatoms selected from N, O or S that is optionally substituted by one or more D; whenever two R⁹ groups occur in the structure then they may optionally together form a 5 or 6 membered heterocyclic ring containing one or more heteroatoms selected from N, O or S, that is optionally substituted by one or more D;
R¹¹ and R¹² are independently selected from hydrogen, halogen, C₁₋₆alkyl, C₂₋₆alkenyl, C₂₋₆alkynyl, C₀₋₆alkylC₃₋₆cycloalkyl, C₀₋₆alkylC₅₋₇cycloalkenyl, C₀₋₆alkylC₆₋₈cycloalkynyl, C₀₋₆alkylaryl, C₀₋₆alkylheterocyclyl and C₀₋₆alkylheteroaryl, wherein said C₁₋₆alkyl, C₂₋₆alkenyl, C₂₋₆alkynyl, C₀₋₆alkylC₃₋₆cycloalkyl, C₀₋₆alkylC₅₋₇cycloalkenyl, C₀₋₆alkylC₆₋₈cycloalkynyl, C₀₋₆alkylaryl, C₀₋₆alkylheteroaryl or C₀₋₆alkylheterocyclyl is optionally substituted by one or more D; or
R¹¹ and R¹² may together form a 4 to 6 membered heterocyclic ring containing one or more heteroatoms selected from N, O or S optionally substituted by one or more D;
D is selected from halogen, nitro, CN, OR¹³, C₁₋₆alkyl, C₂₋₆alkenyl, C₂₋₆alkynyl, C₀₋₆alkylaryl, C₀₋₆alkylheteroaryl, C₀₋₆alkylC₃₋₆cycloalkyl, C₀₋₆alkylC₅₋₇cycloalkenyl, C₀₋₆₋₈alkylC₃₋₆cycloalkynyl, C₀₋₆alkylheterocyclyl, OC₂₋₆alkylNR¹³R¹⁴, NR¹³R¹⁴, CONR¹³R¹⁴, NR¹³(CO)R¹⁴, O(CO)C₁₋₆alkyl, (CO)OC₁₋₆alkyl, COR¹³, (SO₂)NR¹³R¹⁴, NSO₂R¹³, SO₂R¹³SOR¹³, (CO)C₁₋₆alkylNR¹³R¹⁴, (SO₂)C₁₋₆alkylNR¹³R¹⁴, OSO₂R¹³, SO₃R¹³ wherein said C₁₋₆alkyl, C₂₋₆alkenyl, C₂₋₆alkynyl, C₀₋₆alkylaryl, C₀₋₆alkylheteroaryl, C₀₋₆alkylheterocyclyl, C₀₋₆alkylC₃₋₆cycloalkyl, C₀₋₆alkylC₅₋₇cycloalkenyl or C₀₋₆alkylC₆₋₈cycloalkynyl, is optionally substituted with halogen, OSO₂R¹³, SO₃R¹³, nitro, CN, OR¹³, C₁₋₆alkyl;
R¹³ and R¹⁴ are independently selected from hydrogen, halogen, C₁₋₆alkyl, C₃₋₆cycloalkyl, aryl, heteroaryl or heterocyclyl wherein said C₁₋₆alkyl, C₃₋₆cycloalkyl, aryl, heteroaryl or heterocyclyl is optionally substituted by one, two or three hydroxy, CN, halo or C₁₋₃alkyloxy; or
R¹³ and R¹⁴ may together form a 4 to 6 membered heterocyclic ring containing one or more heteroatoms selected from N, O or S optionally substituted by hydroxy, C₁₋₃alkyloxy, cyano or halo;
m=0, 1, 2 or 3;
n=0, 1, 2 or 3;
p=0, 1, 2 or 3;
wherein one of m, n or p is at least 1;
as a free base or a pharmaceutically acceptable salt, solvate or solvate of a salt thereof.

In another aspect of the invention, there is provided a compound of formula I, wherein A is aryl.

In another aspect of the invention, there is provided a compound of formula I, wherein A is phenyl.

In another aspect of the invention, there is provided a compound of formula I, wherein B is aryl.

In another aspect of the invention, there is provided a compound of formula I, wherein B is phenyl.

In another aspect of the invention, there is provided a compound of formula I, wherein C is aryl, substituted with one or more R⁷.

In another aspect of the invention, there is provided a compound of formula I, wherein C is phenyl substituted with one R⁷ and R⁷ represents C₀₋₆alkylOR⁹ and C₀₋₆alkylOR⁹ represents methoxy.

In another aspect of the invention, there is provided a compound of formula I, wherein C is hetoraryl.

In another aspect of the invention, there is provided a compound of formula I, wherein C is pyrimidine.

In another aspect of the invention, there is provided a compound of formula I, wherein C is pyrimidine, substituted with one R⁷ and R⁷ represents methyl or fluoro.

In another aspect of the invention, there is provided a compound of formula I, wherein C is pyrazine.

In another aspect of the invention, there is provided a compound of formula I, wherein C is pyrazole, substituted with one R⁷ and R⁷ represents methyl.

In another aspect of the invention, there is provided a compound of formula I, wherein C is heteroaryl, substituted with one or more R⁷.

In another aspect of the invention, there is provided a compound of formula I, wherein C is pyridine, substituted with one R⁷, said R⁷ being halo. In one embodiment of this aspect, halo represents fluoro. In another embodiment of this aspect, halo represents chloro.

In another aspect of the invention, there is provided a compound of formula I, wherein C is pyridine, substituted with one R⁷, said R⁷ being C₀₋₆alkylOR⁹ and C₀₋₆alkylOR⁹ represents methoxy.

In another aspect of the invention, there is provided a compound of formula I, wherein R¹ is C₁₋₆alkyl. In one embodiment of this aspect, C₁₋₆alkyl is methyl.

In another aspect of the invention, there is provided a compound of formula I, wherein R⁸ is C₁₋₆alkyl. In one embodiment of this aspect, C₁₋₆alkyl is methyl.

In another aspect of the invention, there is provided a compound of formula I, wherein m is 1; n is 0; and p is 0.

In another aspect of the invention, there is provided a compound of formula I, wherein A is aryl; B is aryl; C is aryl or heteroaryl optionally substituted with one or more R⁷; R⁷ is halo or C₀₋₆alkylOR⁹; R⁹ is C₁₋₆alkyl; R¹ is C₁₋₆alkyl; R⁸ is C₁₋₆alkyl; and m is 1; n is 0; and p is 0. In one embodiment of this aspect, A is phenyl; B is phenyl; C is phenyl, pyridine or pyrimidine optionally substituted with one or more R⁷; R⁹ is methyl; R¹ is methyl; and R⁸ is methyl. In another embodiment of this aspect, A is phenyl; B is phenyl; C is phenyl, pyridine, pyrimidine, pyrazine or pyrazole, said phenyl, pyridine, pyrimidine, pyrazine or pyrazole being optionally substituted with one or more R⁷; R⁹ is methyl; R¹ is methyl; and R⁸ is methyl.

In another aspect of the invention, there is provided a compound of formula I, selected from:
2-Amino-5-(3′-methoxybiphenyl-3-yl)-3-methyl-5-[4-(trimethylsilyl)phenyl]-3,5-dihydro-4H-imidazol-4-one;
2-Amino-5-[3-(2-fluoropyridin-3-yl)phenyl]-3-methyl-5-[4-(trimethylsilyl)phenyl]-3,5-dihydro-4H-imidazol-4-one;
2-Amino-3-methyl-5-(3-pyrimidin-5-ylphenyl)-5-[4-(trimethylsilyl)phenyl]-3,5-dihydro-4H-imidazol-4-one;
2-amino-5-[3-(5-methoxypyridin-3-yl)phenyl]-3-methyl-5-(4-trimethylsilylphenyl)imidazol-4-one acetic acid salt;
2-amino-3-methyl-5-(3-pyridin-3-ylphenyl)-5-(4-trimethylsilylphenyl)imidazol-4-one acetic acid salt;
2-amino-5-[3-(5-fluoropyridin-3-yl)phenyl]-3-methyl-5-(4-trimethylsilylphenyl)imidazol-4-one acetic acid salt;
5-[3-[2-amino-1-methyl-5-oxo-4-(4-trimethylsilylphenyl)imidazol-4-yl]phenyl]pyridine-3-carbonitrile acetic acid salt;
2-amino-5-[3-(6-fluoropyridin-3-yl)phenyl]-3-methyl-5-(4-trimethylsilylphenyl)imidazol-4-one acetic acid salt;
3-[3-[2-amino-1-methyl-5-oxo-4-(4-trimethylsilylphenyl)imidazol-4-yl]phenyl]pyridine-4-carbonitrile acetic acid salt;
2-amino-3-methyl-5-[3-(1-methylpyrazol-4-yl)phenyl]-5-(4-trimethylsilylphenyl)imidazol-4-one acetic acid salt;
2-amino-3-methyl-5-[3-(2-methylpyrimidin-5-yl)phenyl]-5-(4-trimethylsilylphenyl)imidazol-4-one acetic acid salt;
2-amino-3-methyl-5-(3-pyrazin-2-ylphenyl)-5-(4-trimethylsilylphenyl)imidazol-4-one;
2-amino-5-[3-(2-fluoropyrimidin-5-yl)phenyl]-3-methyl-5-(4-trimethylsilylphenyl)imidazol-4-one;
2-amino-1-methyl-4-(3-(pyrimidin-5-yl)phenyl)-4-(3-(trimethylsilyl)phenyl)-1H-imidazol-5(4H)-one;
2-amino-4-(3-(5-chloropyridin-3-yl)phenyl)-1-methyl-4-(3-(trimethylsilyl)phenyl)-1H-imidazol-5(4H)-one;
2-amino-4-(3-(6-fluoropyridin-3-yl)phenyl)-1-methyl-4-(3-(trimethylsilyl)phenyl)-1H-imidazol-5(4H)-one;
2-amino-1-methyl-4-(3-(pyridin-3-yl)phenyl)-4-(3-(trimethylsilyl)phenyl)-1H-imidazol-5(4H)-one;
2-amino-4-(3-(2-fluoropyridin-3-yl)phenyl)-1-methyl-4-(3-(trimethylsilyl)phenyl)-1H-imidazol-5(4H)-one;
2-amino-4-(3-(6-methoxypyridin-2-yl)phenyl)-1-methyl-4-(3-(trimethylsilyl)phenyl)-1H-imidazol-5(4H)-one; and
2-amino-1-methyl-4-(3-(pyrazin-2-yl)phenyl)-4-(3-(trimethylsilyl)phenyl)-1H-imidazol-5(4H)-one;
   as a free base or a pharmaceutically acceptable salt, solvate or solvate of a salt thereof.

Some compounds of formula I may have stereogenic centres and/or geometric isomeric centres (E- and Z-isomers), and it is to be understood that the invention encompasses all such optical isomers, enantiomers, diastereoisomers, atropisomers and geometric isomers.

The present invention relates to the use of compounds of formula I as hereinbefore defined as well as to the salts thereof. Salts for use in pharmaceutical compositions will be pharmaceutically acceptable salts, but other salts may be useful in the production of the compounds of formula I.

It is to be understood that the present invention relates to any and all tautomeric forms of the compounds of formula I.

Compounds of the invention can be used as medicaments. In some embodiments, the present invention provides compounds of formula I, or pharmaceutically acceptable salts, tautomers or in vivo-hydrolysable precursors thereof, for use as medicaments. In some embodiments, the present invention provides compounds described here in for use as medicaments for treating or preventing an Aβ-related pathology. In some further embodiments, the Aβ-related pathology is Downs syndrome, a β-amyloid angiopathy, cerebral amyloid angiopathy, hereditary cerebral hemorrhage, a disorder associated with cognitive impairment, MCI (“mild cognitive impairment”), Alzheimer Disease, memory loss, attention deficit symptoms associated with Alzheimer disease, neurodegeneration associated with Alzheimer disease, dementia of mixed vascular origin, dementia of degenerative origin, pre-senile dementia, senile dementia, dementia associated with Parkinson's disease, progressive supranuclear palsy or cortical basal degeneration.

In some embodiments, the present invention provides use of compounds of formula I or pharmaceutically acceptable salts, tautomers or in vivo-hydrolysable precursors thereof, in the manufacture of a medicament for the treatment or prophylaxis of Aβ-related pathologies. In some further embodiments, the Aβ-related pathologies include such as Downs syndrome and β-amyloid angiopathy, such as but not limited to cerebral amyloid angiopathy, hereditary cerebral hemorrhage, disorders associated with cognitive impairment, such as but not limited to MCI (“mild cognitive impairment”), Alzheimer Disease, memory loss, attention deficit symptoms associated with Alzheimer disease, neurodegeneration associated with diseases such as Alzheimer disease or dementia including dementia of mixed vascular and degenerative origin, pre-senile dementia, senile dementia and dementia associated with Parkinson's disease, progressive supranuclear palsy or cortical basal degeneration.

In some embodiments, the present invention provides a method of inhibiting activity of BACE comprising contacting the BACE with a compound of the present invention. BACE is thought to represent the major β-secretase activity, and is considered to be the rate-limiting step in the production of amyloid-β-protein (Aβ). Thus, inhibiting BACE through inhibitors such as the compounds provided herein would be useful to inhibit the deposition of Aβ and portions thereof. Because the deposition of Aβ and portions thereof is linked to diseases such Alzheimer Disease, BACE is an important candidate for the development of drugs as a treatment and/or prophylaxis of Aβ-related pathologies such as Downs syndrome and β-amyloid angiopathy, such as but not limited to cerebral amyloid angiopathy, hereditary cerebral hemorrhage, disorders associated with cognitive impairment, such as but not limited to MCI (“mild cognitive impairment”), Alzheimer Disease, memory loss, attention deficit symptoms associated with Alzheimer disease, neurodegeneration associated with diseases such as Alzheimer disease or dementia including dementia of mixed vascular and degenerative origin, pre-senile dementia, senile dementia and dementia associated with Parkinson's disease, progressive supranuclear palsy or cortical basal degeneration.

In some embodiments, the present invention provides a method for the treatment of Aβ-related pathologies such as Downs syndrome and β-amyloid angiopathy, such as but not limited to cerebral amyloid angiopathy, hereditary cerebral hemorrhage, disorders associated with cognitive impairment, such as but not limited to MCI (“mild cognitive impairment”), Alzheimer Disease, memory loss, attention deficit symptoms associated with Alzheimer disease, neurodegeneration associated with diseases such as Alzheimer disease or dementia including dementia of mixed vascular and degenerative origin, pre-senile dementia, senile dementia and dementia associated with Parkinson's disease, progressive supranuclear palsy or cortical basal degeneration, comprising administering to a mammal (including human) a therapeutically effective amount of a compound of formula I, or a pharmaceutically acceptable salt, tautomer or in vivo-hydrolysable precursor thereof.

In some embodiments, the present invention provides a method for the prophylaxis of Aβ-related pathologies such as Downs syndrome and β-amyloid angiopathy, such as but not limited to cerebral amyloid angiopathy, hereditary cerebral hemorrhage, disorders associated with cognitive impairment, such as but not limited to MCI (“mild cognitive impairment”), Alzheimer Disease, memory loss, attention deficit symptoms associated with Alzheimer disease, neurodegeneration associated with diseases such as Alzheimer disease or dementia including dementia of mixed vascular and degenerative origin, pre-senile dementia, senile dementia and dementia associated with Parkinson's disease, progressive supranuclear palsy or cortical basal degeneration comprising administering to a mammal (including human) a therapeutically effective amount of a compound of formula I or a pharmaceutically acceptable salt, tautomer or in vivo-hydrolysable precursors.

In some embodiments, the present invention provides a method of treating or preventing Aβ-related pathologies such as Downs syndrome and β-amyloid angiopathy, such as but not limited to cerebral amyloid angiopathy, hereditary cerebral hemorrhage, disorders associated with cognitive impairment, such as but not limited to MCI (“mild cognitive impairment”), Alzheimer Disease, memory loss, attention deficit symptoms associated with Alzheimer disease, neurodegeneration associated with diseases such as Alzheimer disease or dementia including dementia of mixed vascular and degenerative origin, pre-senile dementia, senile dementia and dementia associated with Parkinson's disease, progressive supranuclear palsy or cortical basal degeneration by administering to a mammal (including human) a compound of formula I or a pharmaceutically acceptable salt, tautomer or in vivo-hydrolysable precursors and a cognitive and/or memory enhancing agent.

In some embodiments, the present invention provides a method of treating or preventing Aβ-related pathologies such as Downs syndrome and β-amyloid angiopathy, such as but not limited to cerebral amyloid angiopathy, hereditary cerebral hemorrhage, disorders associated with cognitive impairment, such as but not limited to MCI (“mild cognitive impairment”), Alzheimer Disease, memory loss, attention deficit symptoms associated with Alzheimer disease, neurodegeneration associated with diseases such as Alzheimer disease or dementia including dementia of mixed vascular and degenerative origin, pre-senile dementia, senile dementia and dementia associated with Parkinson's disease, progressive supranuclear palsy or cortical basal degeneration by administering to a mammal (including human) a compound of formula I or a pharmaceutically acceptable salt, tautomer or in vivo-hydrolysable precursors thereof wherein constituent members are provided herein, and a choline esterase inhibitor or anti-inflammatory agent.

In some embodiments, the present invention provides a method of treating or preventing Aβ-related pathologies such as Downs syndrome and β-amyloid angiopathy, such as but not limited to cerebral amyloid angiopathy, hereditary cerebral hemorrhage, disorders associated with cognitive impairment, such as but not limited to MCI (“mild cognitive impairment”), Alzheimer Disease, memory loss, attention deficit symptoms associated with Alzheimer disease, neurodegeneration associated with diseases such as Alzheimer disease or dementia including dementia of mixed vascular and degenerative origin, pre-senile dementia, senile dementia and dementia associated with Parkinson's disease, progressive supranuclear palsy or cortical basal degeneration, or any other disease, disorder, or condition described herein, by administering to a mammal (including human) a compound of the present invention and an atypical antipsychotic agent. Atypical antipsychotic agents includes, but not limited to, Olanzapine (marketed as Zyprexa), Aripiprazole (marketed as Abilify), Risperidone (marketed as Risperdal), Quetiapine (marketed as Seroquel), Clozapine (marketed as Clozaril), Ziprasidone (marketed as Geodon) and Olanzapine/Fluoxetine (marketed as Symbyax).

In some embodiments, the mammal or human being treated with a compound of the invention has been diagnosed with a particular disease or disorder, such as those described herein. In these cases, the mammal or human being treated is in need of such treatment. Diagnosis, however, need not be previously performed.

The present invention also includes pharmaceutical compositions which contain, as the active ingredient, one or more of the compounds of the invention herein together with at least one pharmaceutically acceptable carrier, diluent or excipient.

The definitions set forth in this application are intended to clarify terms used throughout this application. The term “herein” means the entire application.

A variety of compounds in the present invention may exist in particular geometric or stereoisomeric forms. The present invention takes into account all such compounds, including cis- and trans isomers, R- and S-enantiomers, diastereomers, (D)-isomers, (L)-isomers, the racemic mixtures thereof, and other mixtures thereof, as being covered within the scope of this invention. Additional asymmetric carbon atoms may be present in a substituent such as an alkyl group. All such isomers, as well as mixtures thereof, are intended to be included in this invention. The compounds herein described may have asymmetric centers. Compounds of the present invention containing an asymmetrically substituted atom may be isolated in optically active or racemic forms. It is well known in the art how to prepare optically active forms, such as by resolution of racemic forms, by synthesis from optically active starting materials, or synthesis using optically active reagents. When required, separation of the racemic material can be achieved by methods known in the art. Many geometric isomers of olefins, C═N double bonds, and the like can also be present in the compounds described herein, and all such stable isomers are contemplated in the present invention. Cis and trans geometric isomers of the compounds of the present invention are described and may be isolated as a mixture of isomers or as separated isomeric forms. All chiral, diastereomeric, racemic forms and all geometric isomeric forms of a structure are intended, unless the specific stereochemistry or isomeric form is specifically indicated.

When a bond to a substituent is shown to cross a bond connecting two atoms in a ring, then such substituent may be bonded to any atom on the ring. When a substituent is listed without indicating the atom via which such substituent is bonded to the rest of the compound of a given formula, then such substituent may be bonded via any atom in such substituent. Combinations of substituents, positions of substituents and/or variables are permissible only if such combinations result in stable compounds.

As used in this application, the term “optionally substituted,” means that substitution is optional and therefore it is possible for the designated atom or moiety to be unsubstituted.

In the event a substitution is desired then such substitution means that any number of hydrogens on the designated atom or moiety is replaced with a selection from the indicated group, provided that the normal valency of the designated atom or moiety is not exceeded, and that the substitution results in a stable compound. For example when a substituent is methyl (i.e., CH₃), then 3 hydrogens on the carbon atom can be replaced. Examples of such substituents include, but are not limited to: halogen, CN, NH₂, OH, COOH, OC₁₋₆alkyl, C₁₋₆alkylOH, SO₂H, C₁₋₆alkyl, OC₁₋₆alkyl, C(O)C₁₋₆alkyl, C(O)OC₁₋₆alkyl, C(O)NH₂, C(O)NHC₁₋₆alkyl, C(O)N(C₁₋₆alkyl)₂, SO₂C₁₋₆alkyl, SO₂NHC₁₋₆alkyl, SO₂N(C₁₋₆alkyl)₂, NH(C₁₋₆alkyl), N(C₁₋₆alkyl)₂, NHC(O)C₁₋₆alkyl, NC(O)(C₁₋₆alkyl)₂, aryl, Oaryl, C(O)aryl, C(O)Oaryl, C(O)NHaryl, C(O)N(aryl)₂, SO₂aryl, SO₂NHaryl, SO₂N(aryl)₂, NH(aryl), N(aryl)₂, NC(O)aryl, NC(O)(aryl)₂, heteroaryl, Oheteroaryl, C(O)heteroaryl, C(O)Oheteroaryl, C(O)NHheteroaryl, C(O)N(heteroaryl)₂, SO₂heteroaryl, SO₂NHheteroaryl, SO₂N(heteroaryl)₂, NH(heteroaryl), N(heteroaryl)₂, NC(O)heteroaryl, NC(O)(heteroaryl)₂, C₅₋₆heterocyclyl, OC₅₋₆heterocyclyl, C(O)C₅₋₆heterocyclyl, C(O)OC₅₋₆heterocyclyl, C(O)NHC₅₋₆heterocyclyl, C(O)N(C₅₋₆heterocyclyl)₂, SO₂C₅₋₆heterocyclyl, SO₂NHC₅₋₆heterocyclyl, SO₂N(C₅₋₆heterocyclyl)₂, NH(C₅₋₆heterocyclyl), N(C₅₋₆heterocyclyl)₂, NC(O)C₅₋₆heterocyclyl, NC(O)(C₅₋₆heterocyclyl)₂.

As used herein, “alkyl”, used alone or as a suffix or prefix, is intended to include both branched and straight chain saturated aliphatic hydrocarbon groups having from 1 to 12 carbon atoms or if a specified number of carbon atoms is provided then that specific number would be intended. For example “C₀₋₆ alkyl” denotes alkyl having 0, 1, 2, 3, 4, 5 or 6 carbon atoms. Examples of alkyl include, but are not limited to, methyl, ethyl, n-propyl, i-propyl, n-butyl, i-butyl, sec-butyl, t-butyl, pentyl, and hexyl. In the case where a subscript is the integer 0 (zero) the group to which the subscript refers to indicates that the group may be absent, i.e. there is a direct bond between the groups.

As used herein, “alkenyl” used alone or as a suffix or prefix is intended to include both branched and straight-chain alkene or olefin containing aliphatic hydrocarbon groups having from 2 to 12 carbon atoms or if a specified number of carbon atoms is provided then that specific number would be intended. For example “C₂₋₆alkenyl” denotes alkenyl having 2, 3, 4, 5 or 6 carbon atoms. Examples of alkenyl include, but are not limited to, vinyl, allyl, 1-propenyl, 1-butenyl, 2-butenyl, 3-butenyl, 2-methylbut-2-enyl, 3-methylbut-1-enyl, 1-pentenyl, 3-pentenyl and 4-hexenyl.

As used herein, “alkynyl” used alone or as a suffix or prefix is intended to include both branched and straight-chain alkyne containing aliphatic hydrocarbon groups having from 2 to 12 carbon atoms or if a specified number of carbon atoms is provided then that specific number would be intended. For example “C₂₋₆alkynyl” denotes alkynyl having 2, 3, 4, 5 or 6 carbon atoms. Examples of alkynyl include, but are not limited to, ethynyl, 1-propynyl, 2-propynyl, 3-butynyl, -pentynyl, hexynyl and 1-methylpent-2-ynyl.

As used herein, the term “cycloalkyl” is intended to include saturated ring groups, having the specified number of carbon atoms. These may include fused or bridged polycyclic systems. Preferred cycloalkyls have from 3 to 10 carbon atoms in their ring structure, and more preferably have 3, 4, 5, and 6 carbons in the ring structure. For example, “C₃₋₆ cycloalkyl” denotes such groups as cyclopropyl, cyclobutyl, cyclopentyl, or cyclohexyl.

As used herein, “cycloalkenyl” is intended to include ring-containing hydrocarbyl groups having at least one carbon-carbon double bond in the ring, and having from 4 to 12 carbons atoms.

As used herein, “cycloalkynyl” is intended to include ring-containing hydrocarbyl groups having at least one carbon-carbon triple bond in the ring, and having from 7 to 12 carbons atoms.

As used herein, “aromatic” refers to hydrocarbonyl groups having one or more unsaturated carbon ring(s) having aromatic characters, (e.g. 4n+2 delocalized electrons) and comprising up to about 14 carbon atoms. In addition “heteroaromatic” refers to groups having one or more unsaturated rings containing carbon and one or more heteroatoms such as nitrogen, oxygen or sulphur having aromatic character (e.g. 4n+2 delocalized electrons).

As used herein, the term “aryl” refers to an aromatic ring structure made up of from 5 to 14 carbon atoms. Ring structures containing 5, 6, 7 and 8 carbon atoms would be single-ring aromatic groups, for example, phenyl. Ring structures containing 8, 9, 10, 11, 12, 13, or 14 would be polycyclic, for example naphthyl. The aromatic ring can be substituted at one or more ring positions with such substituents as described above. The term “aryl” also includes polycyclic ring systems having two or more cyclic rings in which two or more carbons are common to two adjoining rings (the rings are “fused rings”) wherein at least one of the rings is aromatic, for example, the other cyclic rings can be cycloalkyls, cycloalkenyls, cycloalkynyls, aryls and/or heterocyclyls. The terms ortho, meta and para apply to 1,2-, 1,3- and 1,4-disubstituted benzenes, respectively. For example, the names 1,2-dimethylbenzene and ortho-dimethylbenzene are synonymous.

As used herein, “heteroaryl” or “heteroaromatic” refers to an aromatic heterocycle having at least one heteroatom ring member such as sulfur, oxygen, or nitrogen. Heteroaryl groups include monocyclic and polycyclic (e.g., having 2, 3 or 4 fused rings) systems. Examples of heteroaryl groups include without limitation, pyridyl (i.e., pyridinyl), pyrimidinyl, pyrazinyl, pyridazinyl, triazinyl, furyl (i.e. furanyl), quinolyl, isoquinolyl, thienyl, imidazolyl, thiazolyl, indolyl, pyrryl, oxazolyl, benzofuryl, benzothienyl, benzthiazolyl, isoxazolyl, pyrazolyl, triazolyl, tetrazolyl, indazolyl, 1,2,4-thiadiazolyl, isothiazolyl, benzothienyl, purinyl, carbazolyl, fluorenonyl, benzimidazolyl, indolinyl, and the like. In some embodiments, the heteroaryl group has from 1 to about 20 carbon atoms, and in further embodiments from about 3 to about 20 carbon atoms. In some embodiments, the heteroaryl group contains 3 to about 14, 4 to about 14, 3 to about 7, or 5 to 6 ring-forming atoms. In some embodiments, the heteroaryl or heteroaromatic group has 1 to about 4, 1 to about 3, or 1 to 2 heteroatoms. In some embodiments, the heteroaryl or heteroaromatic group has 1 heteroatom.

As used herein, “halo” or “halogen” refers to fluoro, chloro, bromo, and iodo. “Counterion” is used to represent a small, negatively charged species such as chloride, bromide, hydroxide, acetate, sulfate, tosylate, benezensulfonate, and the like.

As used herein, the term “heterocyclyl” or “heterocyclic” or “heterocycle” refers to a saturated, unsaturated or partially saturated, monocyclic, bicyclic or tricyclic ring (unless otherwise stated) containing 3 to 20 atoms of which 1, 2, 3, 4 or 5 ring atoms are chosen from nitrogen, sulphur or oxygen, which may, unless otherwise specified, be carbon or nitrogen linked, wherein a —CH₂— group is optionally be replaced by a —C(O)—; and where unless stated to the contrary a ring nitrogen or sulphur atom is optionally oxidised to form the N-oxide or S-oxide(s) or a ring nitrogen is optionally quarternized; wherein a ring —NH is optionally substituted by acetyl, formyl, methyl or mesyl; and a ring is optionally substituted by one or more halo. It is understood that when the total number of S and O atoms in the heterocyclyl exceeds 1, then these heteroatoms are not adjacent to one another. If the said heterocyclyl group is bi- or tricyclic then at least one of the rings may optionally be a heteroaromatic or aromatic ring provided that at least one of the rings is non-heteroaromatic. If the said heterocyclyl group is monocyclic then it must not be aromatic. Examples of heterocyclyls include, but are not limited to, piperidinyl, N-acetylpiperidinyl, N-methylpiperidinyl, N-formylpiperazinyl, N-mesylpiperazinyl, homopiperazinyl, piperazinyl, azetidinyl, oxetanyl, morpholinyl, tetrahydroisoquinolinyl, tetrahydroquinolinyl, indolinyl, tetrahydropyranyl, dihydro-2H-pyranyl, tetrahydrofuranyl and 2,5-dioxoimidazolidinyl.

As used herein, the phrase “protecting group” means temporary substituents which protect a potentially reactive functional group from undesired chemical transformations. Examples of such protecting groups include esters of carboxylic acids, silyl ethers of alcohols, and acetals and ketals of aldehydes and ketones respectively. The field of protecting group chemistry has been reviewed (Greene, T. W.; Wuts, P. G. M. Protective Groups in Organic Synthesis, 3^{rd} ed.; Wiley: New York, 1999).

As used herein, “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, “pharmaceutically acceptable salts” refer to derivatives of the disclosed compounds wherein the parent compound is modified by making acid or base salts thereof.

Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric acid.

The pharmaceutically acceptable salts of the present invention can be synthesized from the parent compound that contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like diethyl ether, ethyl acetate, ethanol, isopropanol, or acetonitrile are used.

As used herein, “tautomer” means other structural isomers that exist in equilibrium resulting from the migration of a hydrogen atom. For example, keto-enol tautomerism where the resulting compound has the properties of both a ketone and an unsaturated alcohol.

As used herein “stable compound” and “stable structure” are meant to indicate a compound that is sufficiently robust to survive isolation to a useful degree of purity from a reaction mixture, and formulation into an efficacious therapeutic agent.

Compounds of the invention further include hydrates and solvates.

The present invention further includes isotopically-labeled compounds of the invention. An “isotopically” or “radio-labeled” compound is a compound of the invention where one or more atoms are replaced or substituted by an atom having an atomic mass or mass number different from the atomic mass or mass number typically found in nature (i.e., naturally occurring). Suitable radionuclides that may be incorporated in compounds of the present invention include but are not limited to ²H (also written as D for deuterium), ³H (also written as T for tritium), ¹¹C, ¹³C, ¹⁴C, ¹³N, ¹⁵N, ¹⁵O, ¹⁷O, ¹⁸O, ¹⁸F, ³⁵S, ³⁶Cl, ⁸²Br, ⁷⁵Br, ⁷⁶Br, ⁷⁷Br, ¹²³I, ¹²⁴I, ¹²⁵I and ¹³¹I. The radionuclide that is incorporated in the instant radio-labeled compounds will depend on the specific application of that radio-labeled compound. For example, for in vitro receptor labeling and competition assays, compounds that incorporate ³H, ¹⁴C, ⁸²Br, ¹²⁵I, ¹³¹I, ³⁵S or will generally be most useful. For radio-imaging applications ¹¹C, ¹⁸F, ¹²⁵I, ¹²³I, ¹³¹I, ⁷⁵Br, ⁷⁶Br or ⁷⁷Br will generally be most useful.

It is understood that a “radio-labeled compound” is a compound that has incorporated at least one radionuclide. In some embodiments the radionuclide is selected from the group consisting of ³H, ¹⁴C, ¹²⁵I, ³⁵S and ⁸²Br.

The anti-dementia treatment defined herein may be applied as a sole therapy or may involve, in addition to the compound of the invention, conventional chemotherapy. Such chemotherapy may include one or more of the following categories of agents: acetyl cholinesterase inhibitors, anti-inflammatory agents, cognitive and/or memory enhancing agents or atypical antipsychotic agents.

Such conjoint treatment may be achieved by way of the simultaneous, sequential or separate dosing of the individual components of the treatment. Such combination products employ the compounds of this invention.

Compounds of the present invention may be administered orally, parenteral, buccal, vaginal, rectal, inhalation, insufflation, sublingually, intramuscularly, subcutaneously, topically, intranasally, intraperitoneally, intrathoracially, intravenously, epidurally, intrathecally, intracerebroventricularly and by injection into the joints.

The dosage will depend on the route of administration, the severity of the disease, age and weight of the patient and other factors normally considered by the attending physician, when determining the individual regimen and dosage level as the most appropriate for a particular patient.

An effective amount of a compound of the present invention for use in therapy of dementia is an amount sufficient to symptomatically relieve in a warm-blooded animal, particularly a human the symptoms of dementia, to slow the progression of dementia, or to reduce in patients with symptoms of dementia the risk of getting worse.

For preparing pharmaceutical compositions from the compounds of this invention, inert, pharmaceutically acceptable carriers can be either solid or liquid. Solid form preparations include powders, tablets, dispersible granules, capsules, cachets, and suppositories.

A solid carrier can be one or more substances, which may also act as diluents, flavoring agents, solubilizers, lubricants, suspending agents, binders, or tablet disintegrating agents; it can also be an encapsulating material.

In powders, the carrier is a finely divided solid, which is in a mixture with the finely divided active component. In tablets, the active component is mixed with the carrier having the necessary binding properties in suitable proportions and compacted in the shape and size desired.

For preparing suppository compositions, a low-melting wax such as a mixture of fatty acid glycerides and cocoa butter is first melted and the active ingredient is dispersed therein by, for example, stirring. The molten homogeneous mixture is then poured into convenient sized molds and allowed to cool and solidify.

Suitable carriers include magnesium carbonate, magnesium stearate, talc, lactose, sugar, pectin, dextrin, starch, tragacanth, methyl cellulose, sodium carboxymethyl cellulose, a low-melting wax, cocoa butter, and the like.

In some embodiments, the present invention provides a compound of formula I or a pharmaceutically acceptable salt thereof for the therapeutic treatment (including prophylactic treatment) of mammals including humans, it is normally formulated in accordance with standard pharmaceutical practice as a pharmaceutical composition.

In addition to the compounds of the present invention, the pharmaceutical composition of this invention may also contain, or be co-administered (simultaneously or sequentially) with, one or more pharmacological agents of value in treating one or more disease conditions referred to herein.

The term composition is intended to include the formulation of the active component or a pharmaceutically acceptable salt with a pharmaceutically acceptable carrier. For example this invention may be formulated by means known in the art into the form of, for example, tablets, capsules, aqueous or oily solutions, suspensions, emulsions, creams, ointments, gels, nasal sprays, suppositories, finely divided powders or aerosols or nebulisers for inhalation, and for parenteral use (including intravenous, intramuscular or infusion) sterile aqueous or oily solutions or suspensions or sterile emulsions.

Liquid form compositions include solutions, suspensions, and emulsions. Sterile water or water-propylene glycol solutions of the active compounds may be mentioned as an example of liquid preparations suitable for parenteral administration. Liquid compositions can also be formulated in solution in aqueous polyethylene glycol solution. Aqueous solutions for oral administration can be prepared by dissolving the active component in water and adding suitable colorants, flavoring agents, stabilizers, and thickening agents as desired. Aqueous suspensions for oral use can be made by dispersing the finely divided active component in water together with a viscous material such as natural synthetic gums, resins, methyl cellulose, sodium carboxymethyl cellulose, and other suspending agents known to the pharmaceutical formulation art.

The pharmaceutical compositions can be in unit dosage form. In such form, the composition is divided into unit doses containing appropriate quantities of the active component. The unit dosage form can be a packaged preparation, the package containing discrete quantities of the preparations, for example, packeted tablets, capsules, and powders in vials or ampoules. The unit dosage form can also be a capsule, cachet, or tablet itself, or it can be the appropriate number of any of these packaged forms.

Compositions may be formulated for any suitable route and means of administration. Pharmaceutically acceptable carriers or diluents include those used in formulations suitable for oral, rectal, nasal, topical (including buccal and sublingual), vaginal or parenteral (including subcutaneous, intramuscular, intravenous, intradermal, intrathecal and epidural) administration. The formulations may conveniently be presented in unit dosage form and may be prepared by any of the methods well known in the art of pharmacy.

For solid compositions, conventional non-toxic solid carriers include, for example, pharmaceutical grades of mannitol, lactose, cellulose, cellulose derivatives, starch, magnesium stearate, sodium saccharin, talcum, glucose, sucrose, magnesium carbonate, and the like may be used. Liquid pharmaceutically administrable compositions can, for example, be prepared by dissolving, dispersing, etc, an active compound as defined above and optional pharmaceutical adjuvants in a carrier, such as, for example, water, saline aqueous dextrose, glycerol, ethanol, and the like, to thereby form a solution or suspension. If desired, the pharmaceutical composition to be administered may also contain minor amounts of non-toxic auxiliary substances such as wetting or emulsifying agents, pH buffering agents and the like, for example, sodium acetate, sorbitan monolaurate, triethanolamine sodium acetate, sorbitan monolaurate, triethanolamine oleate, etc. Actual methods of preparing such dosage forms are known, or will be apparent, to those skilled in this art; for example, see Remington's Pharmaceutical Sciences, Mack Publishing Company, Easton, Pa., 15th Edition, 1975.

The compounds of the invention may be derivatised in various ways. As used herein “derivatives” of the compounds includes salts (e.g. pharmaceutically acceptable salts), any complexes (e.g. inclusion complexes or clathrates with compounds such as cyclodextrins, or coordination complexes with metal ions such as Mn²⁺ and Zn²⁺), free acids or bases, polymorphic forms of the compounds, solvates (e.g. hydrates), prodrugs or lipids, coupling partners and protecting groups. By “prodrugs” is meant for example any compound that is converted in vivo into a biologically active compound.

Salts of the compounds of the invention are preferably physiologically well tolerated and non toxic. Many examples of salts are known to those skilled in the art. All such salts are within the scope of this invention, and references to compounds include the salt forms of the compounds.

Where the compounds contain an amine function, these may form quaternary ammonium salts, for example by reaction with an alkylating agent according to methods well known to the skilled person. Such quaternary ammonium compounds are within the scope of the invention.

Compounds containing an amine function may also form N-oxides. A reference herein to a compound that contains an amine function also includes the N-oxide.

Where a compound contains several amine functions, one or more than one nitrogen atom may be oxidised to form an N-oxide. Particular examples of N-oxides are the N-oxides of a tertiary amine or a nitrogen atom of a nitrogen-containing heterocycle.

N-Oxides can be formed by treatment of the corresponding amine with an oxidizing agent such as hydrogen peroxide or a per-acid (e.g. a peroxycarboxylic acid), see for example Advanced Organic Chemistry, by Jerry March, 4^{th} Edition, Wiley Interscience, pages. More particularly, N-oxides can be made by the procedure of L. W. Deady (Syn. Comm. 1977, 7, 509-514) in which the amine compound is reacted with m-chloroperoxybenzoic acid (MCPBA), for example, in an inert solvent such as dichloromethane.

Where the compounds contain chiral centres, all individual optical forms such as enantiomers, epimers and diastereoisomers, as well as racemic mixtures of the compounds are within the scope of the invention.

Compounds may exist in a number of different geometric isomeric, and tautomeric forms and references to compounds include all such forms. For the avoidance of doubt, where a compound can exist in one of several geometric isomeric or tautomeric forms and only one is specifically described or shown, all others are nevertheless embraced by the scope of this invention.

The quantity of the compound to be administered will vary for the patient being treated and will vary from about 100 ng/kg of body weight to 100 mg/kg of body weight per day and preferably will be from 10 pg/kg to 10 mg/kg per day. For instance, dosages can be readily ascertained by those skilled in the art from this disclosure and the knowledge in the art. Thus, the skilled artisan can readily determine the amount of compound and optional additives, vehicles, and/or carrier in compositions and to be administered in methods of the invention.

Compounds of the present invention have been shown to inhibit beta secretase (including BACE) activity in vitro. Inhibitors of beta secretase have been shown to be useful in blocking formation or aggregation of Aβ peptide and therefore have beneficial effects in treatment of Alzheimer's Disease and other neurodegenerative diseases associated with elevated levels and/or deposition of Aβ peptide. Therefore, it is believed that the compounds of the present invention may be used for the treatment of Alzheimer disease and disease associated with dementia Hence, compounds of the present invention and their salts are expected to be active against age-related diseases such as Alzheimer, as well as other Aβ related pathologies such as Downs syndrome and β-amyloid angiopathy. It is expected that the compounds of the present invention would most likely be used as single agents but could also be used in combination with a broad range of cognition deficit enhancement agents.

### Methods of Preparation

The present invention also relates to processes for preparing the compound of formula I as a free base or a pharmaceutically acceptable salt thereof. Throughout the following description of such processes it is understood that, where appropriate, suitable protecting groups will be added to, and subsequently removed from the various reactants and intermediates in a manner that will be readily understood by one skilled in the art of organic synthesis. Conventional procedures for using such protecting groups as well as examples of suitable protecting groups are for example described in Protective Groups in Organic Synthesis by T. W. Greene, P. G. M Wutz, 3^{rd} Edition, Wiley-Interscience, New York, 1999. It is understood that microwaves can be used for the heating of reaction mixtures.

### Preparation of Intermediates

The process, wherein A, B, C, D, R¹, R², R³, R⁴, R⁵, R⁶, R⁷, R⁸, R⁹, R¹⁰, R¹¹, R¹², R¹³ and R¹⁴ unless otherwise specified, are as hereinbefore defined, comprises,

(i) Transformation of a compound II to obtain a compound of formula IV through intermediate III, wherein R¹⁵ is as defined hereinbefore for A;

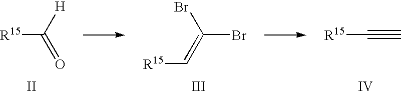

may be carried out by reaction with an appropriately carbon tetra halogen, such as carbon tetra bromide, in the presence of a suitable substituted phosphine such as triphenylphosphine in a suitable halogenated solvent such as dichloromethane. The intermediate corresponding to III is transformed to compound IV with a suitable base such as n-Butyllithium in a suitable solvent such as diethyl ether, THF or Hexane ether or mixtures thereof, at temperatures between −78° C. and RT.

(ii) Cross coupling of a compound of formula V and a compound of formula VI to obtain a compound of formula VII, wherein Halo is a halogen such as bromine, chlorine or iodine, R¹⁵ is as defined hereinbefore for A, and R¹⁶ is as defined hereinbefore for B or trimethylsilyl.

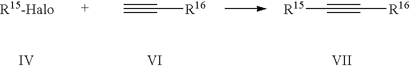

The reaction may be performed with a suitable arylhalide such as a compound of formula II and a suitable alkyne such as (trimethylsilyl)acetylene, in the presence of copper(I) iodide and a suitable palladium catalyst such as dichlorobis(benzonitrile)palladium(II), bis(triphenylphosphine)palladium(II) dichloride, palladium(II) chloride, palladium(0) tetrakistriphenylphosphine with or without a suitable ligand, such as tri-tert-butylphosphine or triphenylphosphine, and a suitable base, such as trietylamine, diisopropylamine or piperidine may be used. The reaction may be performed in a solvent such as tetrahydrofuran or N,N-dimethylformamide, at temperatures between 20° C. and 100° C.

(iii) Desilylation of a compound of formula VIII to a compound of formula IX, wherein R¹⁷ is as defined hereinbefore for A;

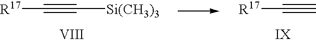

may be performed using silver(I) nitrate or a suitable base such as potassium hydroxide, sodium hydroxide, lithium hydroxide or potassium carbonate, or using a fluoride ion-mediated desilylation using a suitable compound such as tetrabutylammonium fluoride or potassium fluoride. The reaction may be performed in a solvent, such as tetrahydrofuran, methanol, dichloromethane or water, or mixtures thereof, at temperatures between 0° C. and 100° C.

(iv) Oxidation of a compound of formula X to obtain a compound of formula XI, wherein R¹⁸ and R¹⁹ are as defined hereinbefore for A and B respectively;

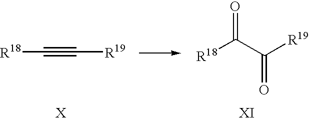

may be performed by reaction with a suitable reagent or mixture of reagents, such as sodium periodate and ruthenium dioxide, iodine and dimethyl sulfoxide, palladium(II) chloride and dimethyl sulfoxide, oxone, hydrogen peroxide, oxygen, potassium permanganate, ruthenium tetroxide or selenium dioxide, in a suitable solvent such as dimethyl sulfoxide, dichloromethane, acetonitrile, water, acetone, chloroform or carbon tetrachloride at a temperature between −78° C. and 150° C. The reaction may be aided by the presence of a catalyst, such as ruthenium(III) chloride or iron(III) chloride.

(v) Conversion of a compound of formula XI to obtain a compound of formula XII, wherein R¹⁸ and R¹⁹ are as defined hereinbefore for A and B respectively, and R¹ is as defined hereinbefore;

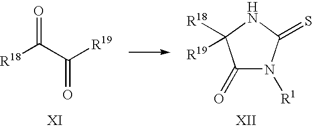

may be carried out by reaction with an appropriately N-substituted thiourea, such as N-methyl thiourea, in the presence of a suitable base such as potassium hydroxide or sodium hydroxide in a suitable solvent, such as water, dimethyl sulfoxide, ethanol or methanol, or mixtures thereof, between 20° C. and reflux.

(vi) Conversion of a compound of formula XII to obtain a compound of formula XIV, wherein R¹⁸, R¹⁹ are as defined hereinbefore for A and B respectively, and R¹ is as defined hereinbefore;

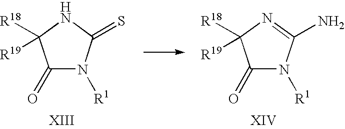

may be carried out by reaction with ammonia, or an ammonia equivalent, together with an alkylhydroperoxide such as t-butylhydroperoxide in a solvent such as ethanol, methanol or water, or a mixture thereof, at 0° C. to 50° C.

(vii) Conversion of a compound of formula XI to obtain a compound of formula XIV, wherein R¹⁸ and R¹⁹ are as defined hereinbefore for A and B respectively, and R¹ is as defined hereinbefore;

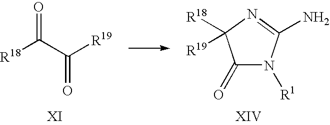

may be carried out by reaction with an appropriately N-substituted urea, such as N-methyl guanidine, in the presence of a suitable base such as sodium carbonate, potassium carbonate or potassium hydroxide in a suitable solvent such as water, dimethyl sulfoxide, dioxane, 2-propanol, ethanol or methanol, or mixtures thereof, between 20° C. and reflux.
Methods of Preparation of End products

Another object of the invention is the process a for the preparation of compounds of general Formula I, wherein A, B, C, D, R¹, R², R³, R⁴, R⁵, R⁶, R⁷, R⁸, R⁹, R¹⁰, R¹¹, R¹², R¹³ and R¹⁴ unless otherwise specified, are defined as hereinbefore, and salts thereof. When it is desired to obtain the acid salt, the free base may be treated with an acid such as a hydrogen halide such as hydrogen chloride in a suitable solvent such as tetrahydrofuran, diethyl ether, methanol, ethanol, chloroform or dichloromethane, or mixtures thereof and the reaction may occur between −30° C. to 50° C.

(a) Conversion of a compound of formula XV to obtain a compound of formula I, wherein Halo represents halogen such as chlorine, bromine or iodine; E is a halogenated formula XIV; C, R⁴ and R⁷ are as defined hereinbefore, and I is defined as hereinbefore;

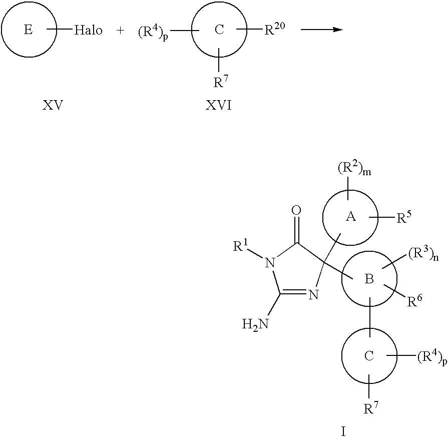

The reaction of process (a) may be carried out by a de-halogen coupling with a suitable compound of formula XV.

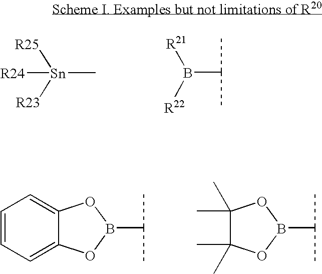

The reaction may be carried out by coupling of a compound of formula XV with an appropriate aryl boronic acid, a boronic ester or a tri-alkylstannyl of formula XVI, wherein R²⁰ may be a group outlined in Scheme I, wherein R²¹ and R²² are groups such as OH, C₁₋₆alkylO or C₂₋₃alkylO and R²¹ and R²² may be fused together to form a 5 or 6 membered boron containing heterocycle and the alkyl, cycloalkyl or aryl moieties may be optionally substituted, wherein R²³, R²⁴ and R²⁵ are groups such as C₁₋₆alkyl. The reaction may be carried out using a suitable palladium catalyst such as tetrakis(triphenylphosphine)palladium(0), palladium diphenylphosphineferrocene dichloride or palladium(II) acetate, together with, or without, a suitable ligand such as tri-tert-butylphosphine or 2-(dicyclohexylphosphino)biphenyl, or using a nickel catalyst such as nickel on charcoal or 1,2-Bis(diphenylphosphino)ethanenickel dichloride together with zinc and sodium triphenylphosphinetrimetasulfonate. A suitable base such as cesium fluoride, an alkyl amine such as triethyl amine, or an alkali metal or alkaline earth metal carbonate or hydroxide such as potassium carbonate, sodium carbonate, cesium carbonate, or sodium hydroxide may be used in the reaction, which may be performed in a temperature range between 20° C., and 160° C., in a suitable solvent such as toluene, tetrahydrofuran, dioxane, dimethoxyethane, water, ethanol or N,N-dimethylformamide, or mixtures thereof.

### General Methods

Starting materials used were available from commercial sources, or prepared according to literature procedures.

Microwave heating was performed in a Creator™, Initiator™ or Smith Synthesizer™ Single-mode microwave cavity producing continuous irradiation at 2450 MHz.

¹H NMR spectra were recorded in the indicated deuterated solvent at 400 MHz. The 400 MHz spectra were obtained unless stated otherwise, using a Bruker DPX400 NMR spectrometer operating at 400 MHz for ¹H, 376 MHz for ¹⁹F, and 100 MHz for ¹³C equipped with a 4-nucleus probehead with Z-gradients. Chemical shifts are given in ppm down- and upfield from TMS. Resonance multiplicities are denoted s, d, t, q, m and br for singlet, doublet, triplet, quartet, multiplet, and broad respectively.

LC-MS analyses were performed on a LC-MS system consisting of a Waters Alliance 2795 HPLC, a Waters PDA 2996 diode array detector, a Sedex 75 ELS detector and a ZQ single quadrupole mass spectrometer. The mass spectrometer was equipped with an electrospray ion source (ES) operated in positive or negative ion mode. The capillary voltage was set to 3.2 kV and the cone voltage to 30 V, respectively. The mass spectrometer was scanned between m/z 100-700 with a scan time of 0.3 s. The diode array detector scanned from 200-400 nm. The temperature of the ELS detector was adjusted to 40° C. and the pressure was set to 1.9 bar. Separation was performed on an X-Terra MS C8, 3.0 mm×50 mm, 3.5 μm (Waters) run at a flow rate of 1 mL/min. A linear gradient was applied starting at 100% A (A: 10 mM ammonium acetate in 5% acetonitrile, or 8 mM formic acid in 5% acetonitrile) ending at 100% B (B: acetonitrile). The column oven temperature was set to 40° C.

GC-MS: Compound identification was performed on a GC-MS system (GC 6890, 5973N MSD) supplied by Agilent Technologies. The column used was a VF-5 MS, ID 0.25 mm×15 m, 0.25 μm (Varian Inc.). A linear temperature gradient was applied starting at 40° C. (hold 1 min) and ending at 300° C. (hold 1 min), 25° C./min. The mass spectrometer was equipped with a chemical ionization (CI) ion source and the reactant gas was methane or the mass spectrometer was equipped with an electron impact (EI) ion source and the electron voltage was set to 70 eV. The mass spectrometer scanned between m/z 50-500 and the scan speed was set to 3.25 scan/s.

Prep-HPLC: Preparative chromatography was run on Waters auto purification HPLC with a diode array detector. Column: XTerra MS C8, 19×300 mm, 10 μm. Gradient with acetonitrile/0.1 M ammonium acetate in 5% acetonitrile in MilliQ Water, typically run from 20% to 60% acetonitrile, in 13 min. Flow rate: 20 mL/min. Alternatively, purification was achieved on a semi preparative Shimadzu LC-8A HPLC with a Shimadzu SPD-10A UV-vis.-detector equipped with a Waters Symmetry® column (C18, 5 μm, 100 mm×19 mm). Gradient with acetonitrile/0.1% trifluoroacetic acid in MilliQ Water, typically run from 35% to 60% acetonitrile in 20 min. Flow rate: 10 mL/min. Alternatively, another column was used; Atlantis C18 19×100 mm, 5 μm column. Gradient with acetonitrile/0.1 M ammonium acetate in 5% acetonitrile in MilliQ Water, run from 0% to 35-50% acetonitrile, in 15 min. Flow rate: 15 mL/min.

Thin layer chromatography (TLC) was performed on Merck TLC-plates (Silica gel 60 F₂₅₄) and spots were UV visualized. Column chromatography was performed using Merck Silica gel 60 (0.040-0.063 mm), or employing a Combi Flash® Companion™ system using RediSep™ normal-phase flash columns.

Compounds have been named using ACD/Name, version 9.0, software from Advanced Chemistry Development, Inc. (ACD/Labs), Toronto ON, Canada, www.acdlabs.com, 2004.

### EXAMPLES

Below follows a number of non-limiting examples of compounds of the invention.

### Example 1

### Trimethyl{4-[(trimethylsilyl)ethynyl]phenyl}silane

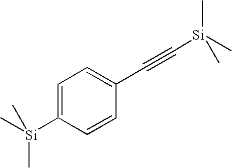

Dichlorobis(benzonitrile)palladium(II) (88 mg, 0.23 mmol), copper(I) iodide (29 mg, 0.15 mmol), tri-tert-butylphosphine (0.9 g, 10 wt % solution in hexane, 0.459 mmol) and diisopropylamine (1.3 mL, 9.19 mmol) were dissolved in anhydrous dioxane (9 mL) under an atmosphere of argon. 1-Bromo-4-(trimethylsilyl)benzene (1.5 mL, 7.66 mmol) and (trimethylsilyl)acetylene (1.27 mL, 9.19 mmol) were added to the above solution and the reaction mixture was stirred overnight. The mixture was diluted with ethyl acetate (40 mL), filtered through a small pad of silica gel, concentrated and purified by column chromatography using n-heptane as the eluent, to give 1.03 g (55% yield) of the title compound: GC-MS (EI) m/z 246 [M]⁺.

### Example 2

### 3-(trimethylsilyl)benzaldehyde

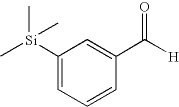

(3-bromophenyl)trimethylsilane (21 g, 91.63 mmol) was dissolved in diethyl ether (250 ml) and cooled to −78° C. n-Butyllithium (47.6 ml, 119.12 mmol) was added and the solution was stirred at −78° C. for 30 min and then at room temp for 90 min. DMF (80 ml) dissolved in diethylether (80 ml) was added and the reaction was stirred at room temp 4 hours. the reaction was quenched by adding 4M HCl (40 ml) and extracted twice with chloroform. the combined organics were washed with NaHCO₃ and brine. solvent was evaporated and crude was purified by flash chromatography 0-30% EtOAc in Heptan to give 3-(trimethylsilyl)benzaldehyde (10 g, 61%). %). ¹H-NMR (DMSO) δ 9.96-10.10 (s, 1H), 8.02-8.07 (s, 1H), 7.87-7.95 (d, 1H), 7.81-7.86 (d, 1H), 7.53-7.63 (t, 1H), 0.24-0.33 (s, 9H) GC-MS (CI) m/z 179 [M+1]⁺

### Example 3

### (4-Ethynylphenyl)(trimethyl)silane

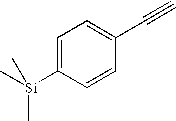

Water (7.65 mL, 419 mmol) and silver nitrate (71 mg, 4.19 mmol) were added to a solution of trimethyl{4-[(trimethylsilyl)ethynyl]phenyl}silane (1.03 g, 4.19 mmol) in acetone (31 mL) and the resulting mixture was protected from light and stirred overnight. It was then poured into a saturated aqueous sodium chloride solution (50 mL) and extracted with diethyl ether (2×40 mL). The organic extract was washed with brine, dried over sodium sulfate and concentrated under reduced pressure to yield 0.63 g (86% yield) of the crude title compound: GC-MS (EI) m/z 174 [M]⁺.

### Example 4

### (3-ethynylphenyl)trimethylsilane

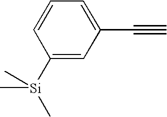

Triphenylphosphine (30.9 g, 117.78 mmol) was dissolved in DCM (50 mL) and cooled to 0° C. Carbon tetrabromide (26.0 g, 78.52 mmol) dissolved in DCM (20 mL) was added and the reaction was stirred for 10 min. 3-(trimethylsilyl)benzaldehyde (7 g, 39.26 mmol) dissolved in DCM (20 mL) was added slowly and the reaction was stirred for 30 min. The reaction was diluted with heptan and filtered thrue a silica plug. The solution was concentrated in vacuo and the resulting intermediate was redissolved in diethyl ether (50 mL) and cooled to −78° C. n-Butyllithium (26.9 mL, 67.34 mmol) was added and the reaction was allowed to reach room temp. stirred for 2 hours and quenched with 3M HCl. Organics were extracted and washed with saturated NaHCO₃ and brine. The solution was concetrated in vacuo to give product (3-ethynylphenyl)trimethylsilane 4.5 g (66.7%). ¹H-NMR (DMSO) δ 7.50-7.56 (s, 1H), 7.31-7.42 (t, 2H), 7.14-7.25 (t, 1H), 2.88-3.05 (s, 1H), 0.15-0.20 (s, 9H); GC-MS (CI) m/z 175 [M+1]⁺

### Example 5

### {4-[(3-Bromophenyl)ethynyl]phenyl}(trimethyl)silane

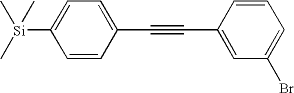

Bis(triphenylphosphine)palladium(II) dichloride (6 mg, 8.10 μmol), (4-ethynylphenyl)(trimethyl)silane (283 mg, 1.62 mmol), copper(I) iodide (2 mg, 8.10 μmol) and triethylamine (1.3 mL) were dissolved in tetrahydrofuran (2 mL). A solution of 1-bromo-3-iodobenzene (0.21 mL, 1.62 mmol) in tetrahydrofuran (2 mL) was added and the resulting mixture was stirred under an atmosphere of argon for 4 h. The mixture was concentrated in vacuo and the residue was re-dissolved in chloroform. The organic layer was washed with water and brine, dried over sodium sulfate and concentrated. The product was purified by column chromatography using n-heptane as eluent, to give 0.44 g (82% yield) of the title compound: ¹H-NMR (CDCl₃) δ 7.74-7.75 (m, 1H), 7.50-7.58 (m, 6H), 7.25-7.31 (m, 1H), 0.34 (s, 9H); ¹³C-NMR (CDCl₃) δ 141.56, 134.32, 133.25, 131.33, 130.69, 130.14, 129.74, 125.33, 122.95, 122.14, 90.84, 88.12, −1.25; GC-MS (EI) m/z 329, 331 [M]⁺.

### Example 6

### {3-[(3-Bromophenyl)ethynyl]phenyl}(trimethyl)silane

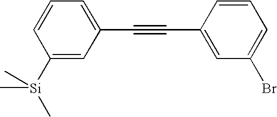

1-bromo-3-iodobenzene (7.30 g, 25.82 mmol), Dichlorobis(triphenylphosphine)-palladium(II) (0.091 g, 0.13 mmol), Copper(I) iodide (0.025 g, 0.13 mmol) and triethylamine (10 mL) were dissolved in tetrahydrofuran (30 mL). A solution of (3-ethynylphenyl)trimethylsilane (4.5 g, 25.82 mmol) in tetrahydrofuran (5 mL) was added and the resulting mixture was stirred under an atmosphere of argon for 4 h. The mixture was concentrated in vacuo and the residue was re-dissolved in chloroform. The organic layer was washed with water and brine, dried over sodium sulfate and concentrated. The product was purified by column chromatography using n-heptane as eluent, to give 8.5 g (94% yield) of the title compound: GC-MS (CI) m/z 329, 331 [M+1]⁺.

### Example 7

### 1-(3-Bromophenyl)-2-[4-(trimethylsilyl)phenyl]ethane-1,2-dione

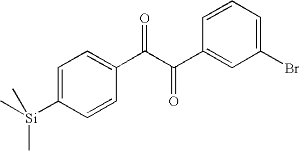

A mixture of {4-[(3-bromophenyl)ethynyl]phenyl}(trimethyl)silane (203 mg, 0.62 mmol) and palladium(II) chloride (11 mg, 0.62 mmol) in dimethyl sulfoxide (3 mL) was stirred overnight at 120° C. Water (7 mL) was added and the aqueous phase was extracted with dichloromethane (20 mL). The organic phase was washed with 1 M hydrochloric acid (2 mL), a saturated aqueous sodium hydrogencarbonate solution, dried over sodium sulfate and concentrated in vacuo. Purification by column chromatography using a gradient of heptane/ethyl acetate as the eluent, to give 151.7 mg (67% yield) of the title compound: ¹³C-NMR (CDCl₃) δ 193.85, 192.92, 150.33, 137.62, 134.73, 133.89, 132.63, 132.47, 130.53, 128.69, 128.52, 123.30, −1.461; MS (ESI) m/z 359 and 361 [M−1]⁻.

### Example 8

### 1-(3-Bromophenyl)-2-[3-(trimethylsilyl)phenyl]ethane-1,2-dione

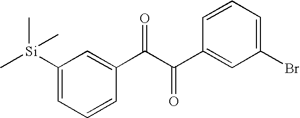

A mixture of (3-((3-bromophenyl)ethynyl)phenyl)trimethylsilane (8 g, 24.29 mmol) and palladium(II) chloride (0.215 g, 1.21 mmol) in dimethyl sulfoxide (40 mL) was stirred overnight at 120° C. Water (80 mL) was added and the aqueous phase was extracted with dichloromethane (40 mL). The organic phase was washed with 1 M hydrochloric acid (20 mL), a saturated aqueous sodium hydrogencarbonate solution, dried over sodium sulfate and concentrated in vacuo. Purification by column chromatography using a gradient of heptane/ethyl acetate as the eluent, to give 8.7 g (87% yield) of the title compound: MS (CI) m/z 363 and 365 [M+1]⁺.

### Example 9

### 5-(3-Bromophenyl)-3-methyl-2-thioxo-5-[4-(trimethylsilyl)phenyl]imidazolidin-4-one

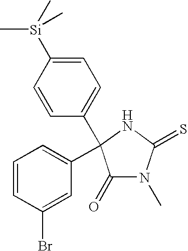

1-(3-Bromophenyl)-2-[4-(trimethylsilyl)phenyl]ethane-1,2-dione (151.70 mg, 0.42 mmol), and N-methylthiourea (76 mg, 0.84 mmol) were dissolved in dimethyl sulfoxide (3 mL). The resulting mixture was heated to 100° C. and potassium hydroxide (1.2 M aqueous solution, 0.72 mL, 0.86 mmol) was added. The reaction mixture was kept at 100° C. for 3 min and then cooled to ambient temperature by addition of ice (20 mL). The resulting mixture was vigorously stirred and the pH was adjusted to around 4 with aqueous hydrochloric acid (2 M). The aqueous phase was extracted with dichloromethane (25 mL), the combined organic phases were washed with a saturated aqueous sodium hydrogencarbonate solution, dried over magnesium sulfate and concentrated in vacuo. Purification by column chromatography using a gradient of heptane/ethyl acetate as the eluent, to give 132.2 mg (68% yield) of the title compound: ¹H NMR (CDCl₃) δ 7.82 (s, 1H), 7.51-7.55 (m, 4H), 7.25-7.29 (m, 4H), 3.33 (s, 3H), 0.27 (s, 9H); MS (ESI) m/z 432 and 434 [M−1]⁻.

### Example 10

### 2-Amino-5-(3-bromophenyl)-3-methyl-5-[4-(trimethylsilyl)phenyl]-3,5-dihydro-4H-imidazol-4-one

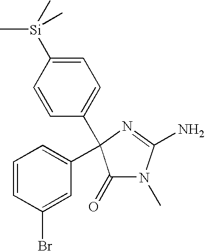

5-(3-Bromophenyl)-3-methyl-2-thioxo-5-[4-(trimethylsilyl)phenyl]imidazolidin-4-one (118.6 mg, 0.27 mmol) was dissolved in methanol (3.8 mL). Aqueous t-butyl hydroperoxide (70%, 0.50 mL, 4.1 mmol) and aqueous ammonia (30%, 1.2 mL) were added and the resulting mixture was stirred at ambient temperature for 6 h. The mixture was concentrated and the resulting residue was re-dissolved in dichloromethane (20 mL). The organic phase was washed with brine, dried over sodium sulfate and concentrated in vacuo. Purification by column chromatography using a gradient of chloroform (containing 0.1% 7 M ammonia in methanol)/methanol as the eluent, to give 103 mg (90% yield) of the title compound: ¹H NMR (CDCl₃) δ 7.67 (t, J=1.8 Hz, 1H), 7.37-7.54 (m, 6H), 7.20 (t, J=8.0 Hz, 1H), 3.21 (s, 3H), 1.28 (s, 1H), 0.25 (s, 9H); MS (ESI) m/z 417 and 419 [M+1]⁺.

### Example 11

### 2-amino-4-(3-bromophenyl)-1-methyl-4-(3-(trimethylsilyl)phenyl)-1H-imidazol-5(4H)-one

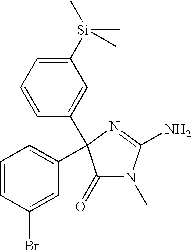

1-(3-bromophenyl)-2-(3-(trimethylsilyl)phenyl)ethane-1,2-dione (9 g, 24.91 mmol) and 1-Methylguanidine hydrochloride (3.55 g, 32.38 mmol) was dissolved in ethanol (50 mL) and dioxane (30 mL) and heated to 50° C. Sodium carbonate (3.96 g, 37.36 mmol) dissolved in water (50.0 mL) was added and the mixture was heated to 80° C. and stirred for 2 hours. volatiles were removed in vacuo and the residue diluted with water and EtOAc. the aqueous phase was removed and the organics were washed once with water and evaporated in vacuo to give 2 g (19.3% yield) of the title compound: ¹H NMR (DMSO) δ 7.57-7.69 (s, 2H), 7.35-7.53 (m, 4H), 7.20-7.35 (q, 2H), 6.59-6.95 (broad s, 2H), 2.92-3.05 (s, 3H), 0.10-0.28 (s, 9H); MS (ES) m/z 414 and 416 [M−1]⁻.

### Example 12

### 2-Amino-5-(3′-methoxybiphenyl-3-yl)-3-methyl-5-[4-(trimethylsilyl)phenyl]-3,5-dihydro-4H-imidazol-4-one

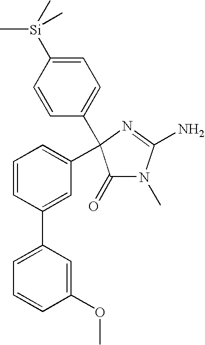

A mixture of 2-amino-5-(3-bromophenyl)-3-methyl-5-[4-(trimethylsilyl)phenyl]-3,5-dihydro-4H-imidazol-4-one (51 mg, 0.12 mmol), 3-methoxybenzeneboronic acid (24 mg, 0.16 mmol), [1,1′-bis(diphenylphosphino)ferrocene]palladium(II) chloride dichloromethane adduct (10 mg, 12.20 μmol) and cesium carbonate (120 mg, 0.37 mmol) in 1,2-dimethoxyethan/water/ethanol (6:3:1, 4 mL) was irradiated in a microwave at 120° C. for 15 min. When cooled to ambient temperature the mixture was diluted with water (5 mL) and the aqueous phase was extracted with dichloromethane. The organic extract was dried over sodium sulfate, concentrated in vacuo and purified by preparative HPLC. The combined HPLC fractions were concentrated, diluted with a saturated aqueous sodium hydrogencarbonate solution and extracted with dichloromethane. The organic layer was dried over sodium sulfate and concentrated in vacuo to give 39.8 mg (73% yield) the title compound: ¹H NMR (CDCl₃) δ 7.91-7.94 (m, 1H), 7.62-7.72 (m, 6H), 7.48-7.58 (m, 2H), 7.26-7.35 (m, 2H), 7.03-7.11 (m, 1H), 4.02 (s, 3H), 3.29 (s, 3H), 0.42 (s, 9H); MS (ESI) m/z 444 [M+1]⁺.

### Example 13

### 2-Amino-5-[3-(2-fluoropyridin-3-yl)phenyl]-3-methyl-5-[4-(trimethylsilyl)phenyl]-3,5-dihydro-4H-imidazol-4-one

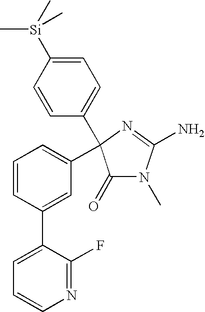

The title compound was synthesized as described for Example 12 in 52% yield, starting from 2-fluoropyridine-3-boronic acid: ¹H NMR (CDCl₃) δ 8.15-8.20 (m, 1H), 7.80-7.87 (m, 1H), 7.70-7.74 (m, 1H), 7.55-7.61 (m, 1H), 7.37-7.52 (m, 6H), 7.21-7.29 (m, 2H), 3.12 (s, 3H), 0.23 (s, 9H); MS (ESI) m/z 433 [M+1]⁺.

### Example 14

### 2-Amino-3-methyl-5-(3-pyrimidin-5-ylphenyl)-5-[4-(trimethylsilyl)phenyl]-3,5-dihydro-4H-imidazol-4-one

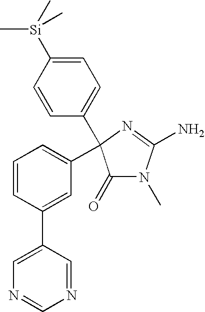

The title compound was synthesized as described for Example 12 in 39% yield, starting from pyrimidin-5-ylboronic acid: ¹H NMR (CDCl₃) δ 9.16 (s, 1H), 8.91 (s, 2H), 7.70-7.78 (m, 1H), 7.58-7.66 (m, 1H), 7.41-7.50 (m, 6H), 3.11 (s, 3H), 0.22 (s, 9H); MS (ESI) m/z 416 [M+1]⁺.

### Example 15

### Chromatographic Preparation of the Enantiomers of 2-amino-3-methyl-5-(3-pyrimidin-5-ylphenyl)-5-[4-(trimethylsilyl)phenyl]-3,5-dihydro-4H-imidazol-4-one

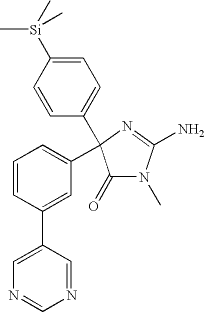

2-Amino-3-methyl-5-(3-pyrimidin-5-ylphenyl)-5-[4-(trimethylsilyl)phenyl]-3,5-dihydro-4H-imidazol-4-one (2.4 g, 4.96 mmol) was dissolved in 2-propanol (150 mL) and the resulting solution was divided into ten equal portions. Chiral separation was carried out on a Chiralpak AD column (50×300 mm), using 2-propanol in heptane (20:80) with 0.1% trifluoroacetic acid as eluent at a flow rate of 120 mL/min, after 20 min the eluent was changed to 100% 2-propanol. The separation was monitored at 254 nm and the two isomers were collected and concentrated in vacuo. The different isomers were converted to hydrochloride using 1 M hydrochloric acid in diethyl ether.

Isomer 1, the first isomer to elute (832 mg, 62% yield): ¹H NMR (CD₃OD) δ 9.17 (s, 1H), 9.05 (s, 2H), 7.82-7.77 (m, 1H), 7.70 (t, J=1.9 Hz, 1H), 7.65-7.56 (m, 3H), 7.52-7.46 (m, 1H), 7.36-7.32 (m, 2H), 3.28 (s, 3H), 0.24 (s, 9H); MS (ES) m/z 416 [M+1]⁺.

Isomer 2, the second isomer to elute (378 mg, 28% yield): ¹H NMR (CD₃OD) δ 9.17 (s, 1H), 9.04 (s, 2H), 7.85-7.78 (m, 1H), 7.72 (t, J=1.9 Hz, 1H), 7.67-7.58 (m, 3H), 7.54-7.48 (m, 1H), 7.37 (d, J=8.6 Hz, 2H), 3.31 (s, 3H), 0.27 (s, 9H); MS (ES) m/z 416 [M+1]⁺.

### Example 16

### 2-amino-5-[3-(5-methoxypyridin-3-yl)phenyl]-3-methyl-5-(4-trimethylsilylphenyl)imidazol-4-one acetic acid salt

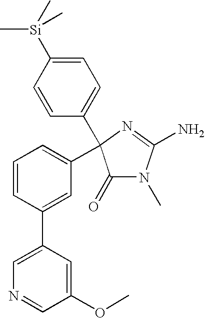

A mixture of 2-amino-5-(3-bromophenyl)-3-methyl-5-[4-(trimethylsilyl)phenyl]-3,5-dihydro-4H-imidazol-4-one (100 mg, 0.24 mmol), 3-methoxy-5-(4,4,5,5-tetramethyl-1,3,2-dioxaborolan-2-yl)pyridine (73 mg, 0.31 mmol), [1,1′-bis(diphenylphosphino)ferrocene]palladium(II) chloride dichloromethane adduct (20 mg, 0.02 mmol) and potassium carbonate (100 mg, 0.72 mmol) in tetrahydrofurane/water (4:1, 2 mL) was irradiated in a microwave reactor at 130° C. for 20 min. When cooled to ambient temperature the organic phase was separated, diluted with methanol (1 mL), filtered and purified by preparative HPLC. The combined HPLC fractions were concentrated under reduced pressure and the remaining aqueous phase was freezedried over night to give 35 mg (32% yield) of the title compound as an acetic acid salt: ¹H NMR (DMSO-d₆) δ ppm 8.35-8.32 (m, 1H) 8.31-8.28 (m, 1H) 7.80-7.75 (m, 1H) 7.62-7.57 (m, 1H) 7.56-7.52 (m, 1H) 7.49-7.41 (m, 6H) 6.75 (br. s., 2H) 3.89 (s, 3H) 2.98 (s, 3H) 1.90 (s, 0.6H) 0.20 (s, 9H); MS (ESI) m/z 445 [M+1]⁺.

### Example 17

### 2-amino-3-methyl-5-(3-pyridin-3-ylphenyl)-5-(4-trimethylsilylphenyl)imidazol-4-one acetic acid salt

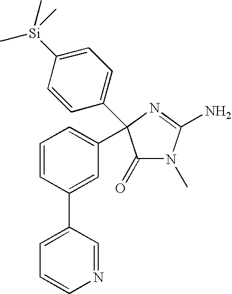

The title compound was synthesized as described for Example 16 in 51% yield, starting from pyridin-3-ylboronic acid: ¹H NMR (DMSO-d₆) δ ppm 8.79-8.75 (m, 1H) 8.60-8.55 (m, 1H) 7.98-7.92 (m, 1H) 7.81-7.77 (m, 1H) 7.61-7.56 (m, 1H) 7.56-7.52 (m, 1H) 7.52-7.41 (m, 6H) 3.01 (s, 3H) 1.91 (s, 1.7H) 0.12 (s, 9H); MS (ESI) m/z 415 [M+1]⁺.

### Example 18

### 2-amino-5-[3-(5-fluoropyridin-3-yl)phenyl]-3-methyl-5-(4-trimethylsilylphenyl)imidazol-4-one acetic acid salt

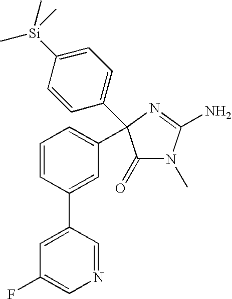

The title compound was synthesized as described for Example 16 in 38% yield, starting from 3-fluoro-5-(4,4,5,5-tetramethyl-1,3,2-dioxaborolan-2-yl)pyridine: ¹H NMR (DMSO-d₆) δ ppm 8.67-8.64 (m, 1H) 8.61-8.58 (m, 1H) 7.95-7.89 (m, 1H) 7.84-7.81 (m, 1 H) 7.66-7.61 (m, 1H) 7.59-7.55 (m, 1H) 7.51-7.42 (m, 5H) 2.99 (s, 3H) 1.89 (s, 2H) 0.18 (s, 9H); MS (ESI) m/z 433 [M+1]⁺.

### Example 19

### 5-[3-[2-amino-1-methyl-5-oxo-4-(4-trimethylsilylphenyl)imidazol-4-yl]phenyl]pyridine-3-carbonitrile acetic acid salt

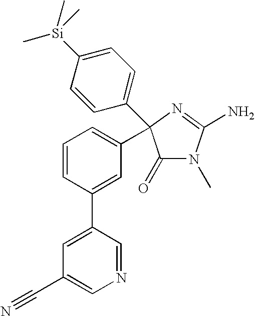

The title compound was synthesized as described for Example 16 in 52% yield, starting from 5-(4,4,5,5-tetramethyl-1,3,2-dioxaborolan-2-yl)pyridine-3-carbonitrile: ¹H NMR (DMSO-d₆) δ ppm 9.06-9.03 (m, 1H) 9.03-9.01 (m, 1H) 8.54-8.50 (m, 1H) 7.88-7.83 (m, 1H) 7.70-7.64 (m, 1H) 7.62-7.57 (m, 1H) 7.54-7.43 (m, 5H) 3.00 (s, 3H) 1.90 (s, 1.85H) 0.11 (s, 9H); MS (ESI) m/z 440 [M+1]⁺.

### Example 20

### 2-amino-5-[3-(6-fluoropyridin-3-yl)phenyl]-3-methyl-5-(4-trimethylsilylphenyl)imidazol-4-one Acetic Acid Salt

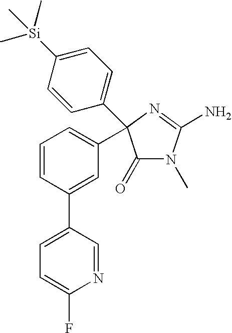

The title compound was synthesized as described for Example 16 in 52% yield, starting from (6-fluoropyridin-3-yl)boronic acid: ¹H NMR (DMSO-d₆) δ ppm 8.42-8.38 (m, 1H) 8.17-8.10 (m, 1H) 7.78-7.74 (m, 1H) 7.58-7.53 (m, 2H) 7.48-7.39 (m, 5H) 7.33-7.21 (m, 1H) 6.71 (br. s., 2H) 2.97 (s, 3H) 1.90 (s, 0.85H) 0.18 (s, 9H); MS (ESI) m/z 433 [M+1]⁺.

### Example 21

### 3-[3-[2-amino-1-methyl-5-oxo-4-(4-trimethylsilylphenyl)imidazol-4-yl]phenyl]pyridine-4-carbonitrile Acetic Acid Salt

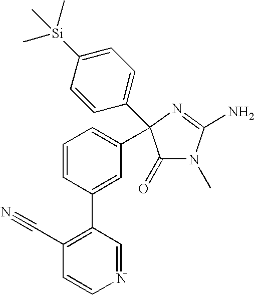

The title compound was synthesized as described for Example 16 in 29% yield, starting from 3-(4,4,5,5-tetramethyl-1,3,2-dioxaborolan-2-yl)pyridine-4-carbonitrile: ¹H NMR (DMSO-d₆) δ ppm 8.88-8.81 (m, 2H) 8.00-7.95 (m, 1H) 7.84-7.78 (m, 1H) 7.66-7.60 (m, 1H) 7.57-7.47 (m, 4H) 7.46-7.42 (m, 2H) 6.79 (br. s., 2H) 2.99 (s, 3H) 1.91 (s, 1.8H) 0.22 (s, 9H); MS (ESI) m/z 440 [M+1]⁺.

### Example 22

### 2-amino-3-methyl-5-[3-(1-methylpyrazol-4-yl)phenyl]-5-(4-trimethylsilylphenyl)imidazol-4-one Acetic Acid Salt

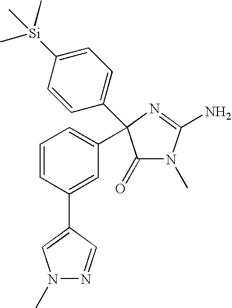

The title compound was synthesized as described for Example 16 in 65% yield, starting from 1-methyl-4-(4,4,5,5-tetramethyl-1,3,2-dioxaborolan-2-yl)pyrazole: ¹H NMR (DMSO-d₆) δ ppm 8.04-7.97 (m, 1H) 7.72-7.68 (m, 1H) 7.64-7.59 (m, 1H) 7.47-7.38 (m, 5H) 7.34-7.24 (m, 2H) 3.85 (s, 3H) 2.99 (s, 3H) 1.88 (s, 6H) 0.20 (s, 9H); MS (ESI) m/z 418 [M+1]⁺.

### Example 23

### 2-amino-3-methyl-5-[3-(2-methylpyrimidin-5-yl)phenyl]-5-(4-trimethylsilylphenyl)imidazol-4-one acetic acid salt

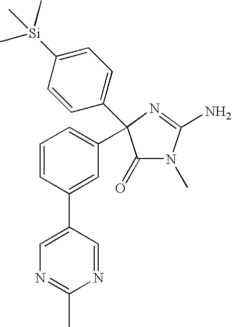

The title compound was synthesized as described for Example 16 in 41% yield, starting from (2-methylpyrimidin-5-yl)boronic acid: ¹H NMR (DMSO-d₆) δ ppm 8.90 (s, 2H) 7.82-7.76 (m, 1H) 7.65-7.59 (m, 1H) 7.59-7.54 (m, 1H) 7.50-7.42 (m, 5H) 2.99 (s, 3H) 2.66 (s, 3H) 1.90 (s, 2H) 0.19 (s, 9H); MS (ESI) m/z 430 [M+1]⁺.

### Example 24

### 2-amino-3-methyl-5-(3-pyrazin-2-ylphenyl)-5-(4-trimethylsilylphenyl)imidazol-4-one

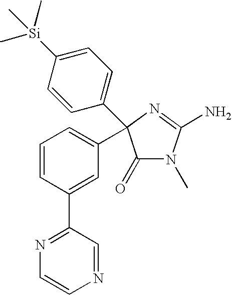

A mixture of 2-amino-5-(3-bromophenyl)-3-methyl-5-[4-(trimethylsilyl)phenyl]-3,5-dihydro-4H-imidazol-4-one (100 mg, 0.24 mmol), tributyl-pyrazin-2-yl-stannane (126 mg, 0.29 mmol) and tetrakis(triphenylphosphine)palladium(0) (28 mg, 0.02 mmol) in dimethylformamide (3 mL) was irradiated in a microwave reactor at 180° C. for 15 min. When cooled to ambient temperature the mixture was diluted with water and extracted with ethyl acetate. The combined organic extracts were dried over sodium sulfate, filtered and concentrated. The resulting residue was dissolved in methanol (3 mL), filtered and purified by preparative HPLC. The combined HPLC fractions were concentrated under reduced pressure and the remaining aqueous phase was diluted with saturated aqueous sodium bicarbonate and extracted with dichloromethane. The combined organics were dried over sodium sulfate, filtered and concentrated to give 25 mg (25% yield) of the title compound: ¹H NMR (DMSO-d₆) δ ppm 9.20-9.14 (m, 1H) 8.74-8.69 (m, 1H) 8.64-8.59 (m, 1H) 8.35-8.29 (m, 1H) 8.02-7.95 (m, 1H) 7.66-7.59 (m, 1H) 7.51-7.42 (m, 5H) 6.73 (br. s., 2H) 2.99 (s, 3H) 0.20 (s, 9H); MS (ESI) m/z 416 [M+1]⁺.

### Example 25

### 2-amino-5-[3-(2-fluoropyrimidin-5-yl)phenyl]-3-methyl-5-(4-trimethylsilylphenyl)imidazol-4-one

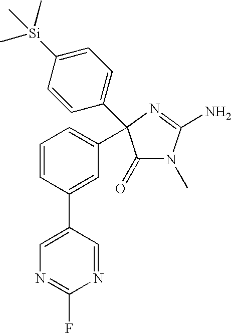

A mixture of 2-amino-5-(3-bromophenyl)-3-methyl-5-[4-(trimethylsilyl)phenyl]-3,5-dihydro-4H-imidazol-4-one (200 mg, 0.48 mmol), Potassium acetate (141 mg, 1.44 mmol), [1,1′-bis(diphenylphosphino)ferrocene]palladium(II) chloride dichloromethane adduct (1:1) (39 mg, 0.05 mmol) and Bis(pinacolato)diboron (134 mg, 0.53 mmol) in 1,2-dimethoxyethane (3 mL) was irradiated in a microwave reactor at 130° C. for 30 min. Then 5-Bromo-2-fluoropyrimidine (85 mg, 0.48 mmol) was added together with water (1 mL) and the mixture run again at 150° C. for 20 min. When cooled to ambient temperature the mixture was diluted with brine, the organic phase separated and the water phase extracted with ethyl acetate. The combined organics were concentrated and purified by preparative HPLC. The combined HPLC fractions were concentrated under reduced pressure and the remaining aqueous phase was diluted with saturated aqueous sodium bicarbonate and extracted with dichloromethane. The combined organics were dried over sodium sulfate, filtered and concentrated to give 25 mg (25% yield) of the title compound; MS (ESI) m/z 434 [M+1]⁺.

### Example 26

### 2-amino-1-methyl-4-(3-(pyrimidin-5-yl)phenyl)-4-(3-(trimethylsilyl)phenyl)-1H-imidazol-5(4H)-one

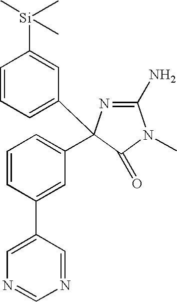

A mixture of 2-amino-5-(3-bromophenyl)-3-methyl-5-[3-(trimethylsilyl)phenyl]-3,5-dihydro-4H-imidazol-4-one (100 mg, 0.24 mmol), Pyrimidine-5-boronic acid (38.7 mg, 0.31 mmol), [1,1′-bis(diphenylphosphino)ferrocene]palladium(II) chloride dichloromethane adduct (19.8 mg, 20 μmol) and potassium carbonate (100 mg, 0.72 mmol) in tetrahydrofuran/water (4:1, 2.5 mL) was irradiated in a microwave at 130° C. for 20 min. When cooled to ambient temperature the aqueous phase was separated and the remaining organic phase was diluted with methanol (1.5 mL) and purified by preparative HPLC. The combined HPLC fractions were freeze dried to give 30 mg (30% yield) the title compound: ¹H NMR (DMSO) δ 9.13-9.28 (s, 1H), 8.91-9.05 (s, 2H), 7.76-7.83 (s, 1H), 7.69-7.74 (s, 1H), 7.63-7.68 (d, 1H), 7.54-7.60 (d, 1H), 7.44-7.54 (m, 2H), 7.35-7.43 (d, 1H), 7.25-7.34 (t, 1H), 3.00 (s, 3H), 0.08-0.33 (s, 9H); MS (ESI) m/z 416 [M+1]⁺.

### Example 27

### 2-amino-4-(3-(5-chloropyridin-3-yl)phenyl)-1-methyl-4-(3-(trimethylsilyl)phenyl)-1H-imidazol-5(4H)-one

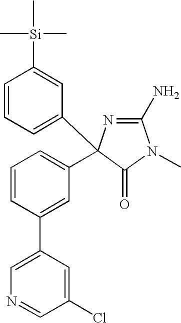

The title compound was synthesized as described for Example 26 in 65% yield, starting from 5-chloropyridin-3-ylboronic acid: ¹H NMR (DMSO) δ 8.67-8.74 (s, 1H), 8.59-8.66 (s, 1H), 8.02-8.11 (s, 1H), 7.76-7.83 (s, 1H), 7.69-7.74 (s, 1H), 7.63-7.68 (d, 1H), 7.54-7.60 (d, 1H), 7.41-7.53 (m, 2H), 7.34-7.41 (d, 1H), 7.25-7.33 (t, 1H), 3.00 (s, 3H), 0.08-0.33 (s, 9H); MS (ESI) m/z 450 [M+1]⁺.

### Example 28

### 2-amino-4-(3-(6-fluoropyridin-3-yl)phenyl)-1-methyl-4-(3-(trimethylsilyl)phenyl)-1H-imidazol-5(4H)-one

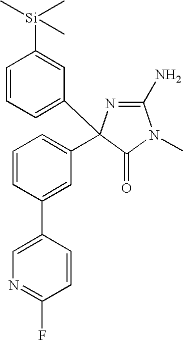

The title compound was synthesized as described for Example 26 in 15.6% yield, starting from 6-fluoropyridin-3-ylboronic acid: ¹H NMR (DMSO) δ 8.33-8.47 (s, 1H), 8.02-8.14 (dt, 1H), 7.72-7.77 (s, 1H), 7.66-7.71 (s, 1H), 7.47-7.60 (m, 3H), 7.40-7.47 (t, 1H), 7.34-7.40 (d, 1H), 7.24-7.32 (m, 2H), 3.00 (s, 3H), 0.06-0.30 (s, 9H); MS (ESI) m/z 431 [M−1]⁻.

### Example 29

### 2-amino-1-methyl-4-(3-(pyridin-3-yl)phenyl)-4-(3-(trimethylsilyl)phenyl)-1H-imidazol-5(4H)-one

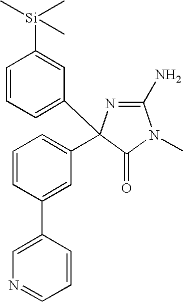

The title compound was synthesized as described for Example 26 in 17.2% yield, starting from pyridin-3-ylboronic acid: ¹H NMR (DMSO) δ 8.71-8.79 (s, 1H), 8.52-8.65 (d, 1H), 7.89-7.97 (d, 1H), 7.74-7.81 (s, 1H), 7.67-7.73 (s, 1H), 7.41-7.61 (m, 5H), 7.35-7.41 (d, 1H), 7.25-7.34 (t, 1H), 3.00 (s, 3H), 0.08-0.28 (s, 9H); MS (ESI) m/z 413 [M−1]⁻.

### Example 30

### 2-amino-4-(3-(2-fluoropyridin-3-yl)phenyl)-1-methyl-4-(3-(trimethylsilyl)phenyl)-1H-imidazol-5(4H)-one

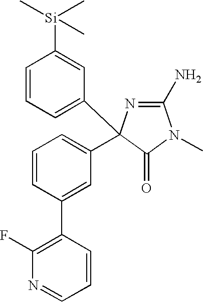

The title compound was synthesized as described for Example 26 in 15.2% yield, starting from 2-fluoropyridin-3-ylboronic acid: ¹H NMR (DMSO) δ 8.19-8.27 (d, 1H), 7.93-8.03 (t, 1H), 7.73-7.77 (s, 1H), 7.67-7.72 (s, 1H), 7.34-7.59 (m, 6H), 7.24-7.33 (t, 1H), 3.00 (s, 3H), 0.09-0.30 (s, 9H); MS (ESI) m/z 431 [M−1]⁻.

### Example 31

### 2-amino-4-(3-(6-methoxypyridin-2-yl)phenyl)-1-methyl-4-(3-(trimethylsilyl)phenyl)-1H-imidazol-5(4H)-one

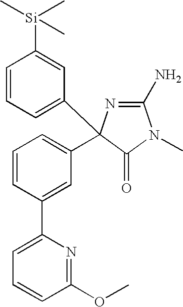

The title compound was synthesized as described for Example 26 in 22.5% yield, starting from 2-methoxy-6-(4,4,5,5-tetramethyl-1,3,2-dioxaborolan-2-yl)pyridine: ¹H NMR (DMSO) δ 8.16-8.25 (s, 1H), 7.87-7.95 (d, 1H), 7.71-7.81 (m, 2H), 7.50-7.57 (d, 1H), 7.35-7.50 (m, 4H), 7.27-7.34 (t, 1H), 6.74-6.79 (d, 1H), 3.90 (s, 3H), 3.00 (s, 3H), 0.12-0.30 (s, 9H); MS (ESI) m/z 443 [M−1]⁻.

### Example 32

### 2-amino-1-methyl-4-(3-(pyrazin-2-yl)phenyl)-4-(3-(trimethylsilyl)phenyl)-1H-imidazol-5(4H)-one

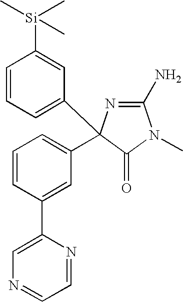

2-amino-4-(3-bromophenyl)-1-methyl-4-(3-(trimethylsilyl)phenyl)-1H-imidazol-5(4H)-one (100 mg, 0.24 mmol), 2-(tributylstannyl)pyrazine (106 mg, 0.29 mmol) and Tetrakis(triphenylphosphine)palladium(0) (28 mg, 0.02 mmol) in DMF (3 ml) were irradiated in a microwave reactor at 180° C. for 15 minutes. When cooled to ambient temperature the mixture was diluted with water and extracted with dichloromethane. The organic phase was evaporated and the resulting residue was redissolved in methanol and purified by preparative HPLC, combined fractions were freeze dried to give 2-amino-1-methyl-4-(3-(pyrazin-2-yl)phenyl)-4-(3-(trimethylsilyl)phenyl)-1H-imidazol-5(4H)-one (5 mg, 5.0%): ¹H NMR (DMSO) δ 9.10-9.20 (s, 1H), 8.69-8.79 (s, 1H), 8.56-8.64 (s, 1H), 8.27-8.34 (s, 1H), 7.94-8.02 (d, 1H), 7.66-7.77 (s, 1H), 7.56-7.64 (d, 1H), 7.42-7.52 (m, 2H), 7.35-7.42 (d, 1H), 7.25-7.34 (t, 1H), 2.97-3.03 (s, 3H), 0.09-0.30 (s, 9H); MS (ESI) m/z 416 [M+1]⁺.

### Assays

Compounds were tested in at least one of the following assays:

\tilde{␣}Secretase Enzyme

The enzyme used in the IGEN Cleavage-, Fluorescent-, TR-FRET- and the BiaCore assay is described as follows:

The soluble part of the human β-Secretase (AA 1-AA 460) was cloned into the ASP2-Fc10-1-IRES-GFP-neoK mammalian expression vector. The gene was fused to the Fc domain of IgG1 (affinity tag) and stably cloned into HEK 293 cells. Purified sBACE-Fc is stored in Tris buffer, pH 9.2 and has a purity of 95%.

### IGEN Cleavage Assay

Enzyme is diluted 1:30 in 40 mM MES pH 5.0. Stock substrate is diluted to 12 μM in 40 mM MES pH 5.0. Compounds are diluted to the desired concentration in dimethylsulphoxide (final dimethylsulphoxide concentration in assay is 5%). The assay is done in a 96 well PCR plate from Greiner (#650201). Compound in dimethylsulphoxide (3 μL) is added to the plate, and then enzyme is added (27 μL) and pre-incubated with compound for 10 minutes. The reaction is started with substrate (30 μL). The final dilution of enzyme is 1:60 and the final concentration of substrate is 6 μM. After a 20 minute reaction at room temperature, the reaction is stopped by removing 10 μl of the reaction mix and diluting it 1:25 in 0.2 M Trizma-HCl, pH 8.0. Compounds are diluted and added to the plate by the Biomek FX or by hand, then all the rest of the liquid handling is done with on the Biomek 2000 instrument.

All antibodies and the streptavidin coated beads are diluted in PBS containing 0.5% BSA and 0.5% Tween20. The product is quantified by adding 50 μL of a 1:5000 dilution of the neoepitope antibody to 50 μL of the 1:25 dilution of the reaction mix. Then, 100 μL of PBS (0.5% BSA, 0.5% Tween20) containing 0.2 mg/mL IGEN beads (Dynabeads M-280) and a 1:5000 dilution of ruthinylated goat anti-rabbit (Ru-GαR) antibody is added. The final dilution of neoepitope antibody is 1:20,000, the final dilution of Ru-GAR is 1:10,000 and the final concentration of beads is 0.1 mg/mL. The mixture is read on the IGEN M8 Analyzer (BioVeris) after 2-hour incubation with shaking at room temperature. The dimethylsulphoxide control defines 100% activity level and 0% activity is defined by exclusion of the enzyme (using 40 mM MES pH 5.0 buffer instead).

### Fluorescent Assay

Enzyme is diluted 1:25 in 40 mM MES pH 5.0. Stock substrate (Dabcyl) is diluted to 30 μM in 40 mM MES pH 5.0. Enzyme and substrate stock solutions are kept on ice until placed in the stock plates. The Biomek FX instrument is used to do all liquid handling. Enzyme (9 μL) together with 1 μL of compound in dimethylsulphoxide is added to the plate and pre-incubated for 10 minutes. When a dose response curve is being tested for a compound, the dilutions are done in neat dimethylsulphoxide. Substrate (10 μL) is added and the reaction proceeds in the dark for 25 minutes at room temperature. The assay is done in a Corning 384 well round bottom, low volume, non-binding surface (Corning #3676). The final dilution of enzyme is 1:50, and the final concentration of substrate is 15 μM (Km of 25 μM). The fluorescence of the product is measured on a Wallac Victor TI plate reader with an excitation wavelength of 360 nm and an emission wavelength of 485 nm using the protocol for labelled Edans peptide. The dimethylsulphoxide control defines 100% activity level and 0% activity is defined by exclusion of the enzyme (using 40 mM MES pH 5.0 buffer instead).

### TR-FRET Assay

Dilute the enzyme (truncated form) to 6 μg/mL (stock 1.3 mg/mL) and the substrate (Europium)CEVNLDAEFK(Qsy7) to 200 nM (stock 60 μM) in reaction buffer (NaAcetate, chaps, triton x-100, EDTA pH4.5). The Biomek FX is used for all liquid handling and the enzyme and substrate solutions are kept on ice until they are placed in Biomek FX. Enzyme (9 μl) is added to the plate then 1 μl of compound in dimethylsulphoxide is added, mixed and pre-incubated for 10 minutes. Substrate (10 μl) is then added, mixed and the reaction proceeds in the dark for 15 minutes at room temperature. The reaction is stopped with the addition of Stop solution (7 μl, NaAcetate pH 9). The fluorescence of the product is measured on a Wallac Victor II plate reader with an excitation wavelength of 340 nm and an emission wavelength of 615 nm. The assay is done in a Costar 384 well round bottom, low volume, non-binding surface (Corning #3676). The final concentration of the enzyme is 0.3 nM; the final concentration of substrate is 100 nM (Km of ˜250 nM). The dimethylsulphoxide control defines the 100% activity level and 0% activity is defined by exclusion of the enzyme, only using reaction buffer instead.

### Beta-Secretase Whole Cell Assay

### Generation of HEK293-APP695

The pcDNA3.1 plasmid encoding the cDNA of human full-length APP695 was stably transfected into HEK-293 cells using the Lipofectamine transfection reagent according to manufacture's protocol (Invitrogen). Colonies were selected with 0.1-0.5 mg/mL of zeocin. Limited dilution cloning was performed to generate homogeneous cell lines. Clones were characterized by levels of APP expression and Aβ secreted in the conditioned media using an ELISA assay developed in-house.

### Cell Culture

HEK293 cells stably expressing human wild-type APP (HEK293-APP695) were grown at 37° C. in DMEM containing 4500 g/L glucose, GlutaMAX and sodium pyruvate supplemented with 10% FBS, 1% non-essential amino acids and 0.1 mg/mL of the selection antibiotic zeocin.

### Aβ840 Release Assay

Cells were harvested at 80-90% confluence and seeded at a concentration of 0.2×10⁶ cells/mL, 100 mL cell suspension/well, onto a black clear bottom 96-well poly-D-lysine coated plate. After over night incubation at 37° C., 5% CO₂, the cell medium was replaced with cell culture medium with penicillin and streptomycin (100 U/mL, 100 μg/mL, respectively) containing test compounds in a final dimethylsulphoxide concentration of 1%. Cells were exposed to test compounds for 24 h at 37° C., 5% CO2. To quantify the amount of released Aβ, 100 μL cell medium was transferred to a round bottom polypropylene 96-well plate (assay plate). The cell plate was saved for ATP assay as described in ATP assay below. To the assay plate, 50 μL of primary detection solution containing 0.5 μg/mL of rabbit anti-Aβ40 antibody and 0.5 μg/mL of biotinylated monoclonal mouse 6E10 antibody in DPBS with 0.5% BSA and 0.5% Tween-20 was added per well and incubated over night at 4° C. Then, 50 μL of secondary detection solution containing 0.5 μg/mL of a ruthenylated goat anti-rabbit antibody and 0.2 mg/mL of streptavidin coated Dynabeads was added per well. The plate was vigorously shaken at room temperature for 1-2 h. The plate was then measured for electro-chemiluminescence counts in an IGEN M8 Analyzer (BioVeris). An Aβ standard curve was obtained using standards at concentrations 20, 10, 2 and 0.2 ng Aβ/mL in the cell culture medium with penicillin and streptomycin (100 U/mL, 100 μg/mL, respectively).

ATP assay

As indicated above, after transferring 100 μL medium from the cell plate for A040 detection, the plate was used to analyse cytotoxicity using the ViaLight™ Plus cell proliferation/cytotoxicity kit from Cambrex BioScience that measures total cellular ATP. The assay was performed according to the manufacture's protocol. Briefly, 50 μL cell lysis reagent was added per well. The plates were incubated at room temperature for 10 min. Two min after addition of 100 μL reconstituted ViaLight™ Plus ATP reagent, the luminescence was measured in a Wallac Victor² 1420 multilabel counter.

### BACE Biacore Protocol

### Sensor Chip Preparation:

BACE was assayed on a Biacore3000 instrument by attaching either a peptidic transition state isostere (TSI) or a scrambled version of the peptidic TSI to the surface of a Biacore CM5 sensor chip. The surface of a CM5 sensor chip has 4 distinct channels that can be used to couple the peptides. The scrambled peptide KFES-statine-ETIAEVENV was coupled to channel 1 and the TSI inhibitor KTEEISEVN-statine-VAEF was couple to channel 2 of the same chip. The two peptides were dissolved at 0.2 mg/mL in 20 mM Na-Acetate pH 4.5, and then the solutions were centrifuged at 14K rpm to remove any particulates. Carboxyl groups on the dextran layer were activated by injecting a one to one mixture of 0.5 M N-ethyl-N′(3-dimethylaminopropyl)-carbodiimide (EDC) and 0.5 M N-hydroxysuccinimide (NHS) at 5 uL/minute for 7 minutes. Then the stock solution of the control peptide was injected in channel 1 for 7 minutes at 5 uL/min., and then the remaining activated carboxyl groups were blocked by injecting 1M ethanolamine for 7 minutes at 5 uL/minute.

### Assay Protocol

The BACE Biacore assay was done by diluting BACE to 0.5 μM in Na Acetate buffer at pH 4.5 (running buffer minus dimethylsulphoxide). The diluted BACE was mixed with dimethylsulphoxide or compound diluted in dimethylsulphoxide at a final concentration of 5% dimethylsulphoxide. The BACE/inhibitor mixture was incubated for 1 hour at 4° C. then injected over channel 1 and 2 of the CM5 Biacore chip at a rate of 20 μL/minute. As BACE bound to the chip the signal was measured in response units (RU). BACE binding to the TSI inhibitor on channel 2 gave a certain signal. The presence of a BACE inhibitor reduced the signal by binding to BACE and inhibiting the interaction with the peptidic TSI on the chip. Any binding to channel 1 was non-specific and was subtracted from the channel 2 responses. The dimethylsulphoxide control was defined as 100% and the effect of the compound was reported as percent inhibition of the dimethylsulphoxide control.

hERG Assay

### Cell Culture

The hERG-expressing Chinese hamster ovary K1 (CHO) cells described by (Persson, Carlsson, Duker, & Jacobson, 2005) were grown to semi-confluence at 37° C. in a humidified environment (5% CO₂) in F-12 Ham medium containing L-glutamine, 10% foetal calf serum (FCS) and 0.6 mg/ml hygromycin (all Sigma-Aldrich). Prior to use, the monolayer was washed using a pre-warmed (37° C.) 3 ml aliquot of Versene 1:5,000 (Invitrogen). After aspiration of this solution the flask was incubated at 37° C. in an incubator with a further 2 ml of Versene 1:5,000 for a period of 6 minutes. Cells were then detached from the bottom of the flask by gentle tapping and 10 ml of Dulbecco's Phosphate-Buffered Saline containing calcium (0.9 mM) and magnesium (0.5 mM) (PBS; Invitrogen) was then added to the flask and aspirated into a 15 ml centrifuge tube prior to centrifugation (50 g, for 4 mins). The resulting supernatant was discarded and the pellet gently re-suspended in 3 ml of PBS. A 0.5 ml aliquot of cell suspension was removed and the number of viable cells (based on trypan blue exclusion) was determined in an automated reader (Cedex; Innovatis) so that the cell re-suspension volume could be adjusted with PBS to give the desired final cell concentration. It is the cell concentration at this point in the assay that is quoted when referring to this parameter. CHO-Kv1.5 cells, which were used to adjust the voltage offset on IonWorks™ HT, were maintained and prepared for use in the same way.

### Electrophysiology

The principles and operation of this device have been described by (Schroeder, Neagle, Trezise, & Worley, 2003). Briefly, the technology is based on a 384-well plate (PatchPlate™) in which a recording is attempted in each well by using suction to position and hold a cell on a small hole separating two isolated fluid chambers. Once sealing has taken place, the solution on the underside of the PatchPlate™ is changed to one containing amphotericin B. This permeablises the patch of cell membrane covering the hole in each well and, in effect, allows a perforated, whole-cell patch clamp recording to be made.

A □-test IonWorks™ HT from Essen Instrument was used. There is no capability to warm solutions in this device hence it was operated at room temperature (˜21° C.), as follows. The reservoir in the “Buffer” position was loaded with 4 ml of PBS and that in the “Cells” position with the CHO-hERG cell suspension described above. A 96-well plate (V-bottom, Greiner Bio-one) containing the compounds to be tested (at 3-fold above their final test concentration) was placed in the “Plate 1” position and a PatchPlate™ was clamped into the PatchPlate™ station. Each compound plate was laid-out in 12 columns to enable ten, 8-point concentration-effect curves to be constructed; the remaining two columns on the plate were taken up with vehicle (final concentration 0.33% DMSO), to define the assay baseline, and a supra-maximal blocking concentration of cisapride (final concentration 10 □M) to define the 100% inhibition level. The fluidics-head (F-Head) of IonWorks™ HT then added 3.5 μl of PBS to each well of the PatchPlate™ and its underside was perfused with “internal” solution that had the following composition (in mM): K-Gluconate 100, KCl 40, MgCl₂ 3.2, EGTA 3 and HEPES 5 (all Sigma-Aldrich; pH 7.25-7.30 using 10 M KOH). After priming and de-bubbling, the electronics-head (E-head) then moved round the PatchPlate™ performing a hole test (i.e. applying a voltage pulse to determine whether the hole in each well was open). The F-head then dispensed 3.5 μl of the cell suspension described above into each well of the PatchPlate™ and the cells were given 200 seconds to reach and seal to the hole in each well. Following this, the E-head moved round the PatchPlate™ to determine the seal resistance obtained in each well. Next, the solution on the underside of the PatchPlate™ was changed to “access” solution that had the following composition (in mM): KCl 140, EGTA 1, MgCl₂ 1 and HEPES 20 (pH 7.25-7.30 using 10 M KOH) plus 100 □g/ml of amphotericin B (Sigma-Aldrich). After allowing 9 minutes for patch perforation to take place, the E-head moved round the PatchPlate™ 48 wells at a time to obtain pre-compound hERG current measurements. The F-head then added 3.5 ␣l of solution from each well of the compound plate to 4 wells on the PatchPlate™ (the final DMSO concentration was 0.33% in every well). This was achieved by moving from the most dilute to the most concentrated well of the compound plate to minimise the impact of any compound carry-over. After approximately 3.5 mins incubation, the E-head then moved around all 384-wells of the PatchPlate™ to obtain post-compound hERG current measurements. In this way, non-cumulative concentration-effect curves could be produced where, providing the acceptance criteria were achieved in a sufficient percentage of wells (see below), the effect of each concentration of test compound was based on recording from between 1 and 4 cells.

The pre- and post-compound hERG current was evoked by a single voltage pulse consisting of a 20 s period holding at −70 mV, a 160 ms step to −60 mV (to obtain an estimate of leak), a 100 ms step back to −70 mV, a 1 s step to +40 mV, a 2 s step to −30 mV and finally a 500 ms step to −70 mV. In between the pre- and post-compound voltage pulses there was no clamping of the membrane potential. Currents were leak-subtracted based on the estimate of current evoked during the +10 mV step at the start of the voltage pulse protocol. Any voltage offsets in IonWorks™ HT were adjusted in one of two ways. When determining compound potency, a depolarising voltage ramp was applied to CHO-Kv1.5 cells and the voltage noted at which there was an inflection point in the current trace (i.e. the point at which channel activation was seen with a ramp protocol). The voltage at which this occurred had previously been determined using the same voltage command in conventional electrophysiology and found to be −15 mV (data not shown); thus an offset potential could be entered into the IonWorks™ HT software using this value as a reference point. When determining the basic electrophysiological properties of hERG, any offset was adjusted by determining the hERG tail current reversal potential in IonWorks™ HT, comparing it with that found in conventional electrophysiology (−82 mV) and then making the necessary offset adjustment in the IonWorks™ HT software. The current signal was sampled at 2.5 kHz.

Pre- and post-scan hERG current magnitude was measured automatically from the leak subtracted traces by the IonWorks™ HT software by taking a 40 ms average of the current during the initial holding period at −70 mV (baseline current) and subtracting this from the peak of the tail current response. The acceptance criteria for the currents evoked in each well were: pre-scan seal resistance >60 MΩ, pre-scan hERG tail current amplitude >150 pA; post-scan seal resistance >60 MΩ. The degree of inhibition of the hERG current was assessed by dividing the post-scan hERG current by the respective pre-scan hERG current for each well.

### Results

Typical IC50 values for the compounds of the present invention are in the range of about 1 to about 10,000 nM. Biological data on final compounds are given below in Table 1.

TABLE 1

| Example No. | IC50 (nM) in TR-FRET assay |
|---|---|
| 12 | 84 |
| 13 | 81 |
| 14 | 107 (racemate) |
| 15 | 41 (isomer 1) |
| | >10.000 (isomer 2) |
| 16 | 23 |
| 17 | 118 |
| 18 | 180 |
| 19 | 118 |
| 20 | 437 |
| 21 | 460 |
| 22 | 2742 |
| 23 | 2260 |
| 24 | 80 |
| 25 | 460 |
| 26 | 135 |
| 27 | 163 |
| 28 | 278 |
| 29 | 202 |
| 30 | 182 |
| 31 | 1530 |
| 32 | 86 |

1. A compound according to formula I: 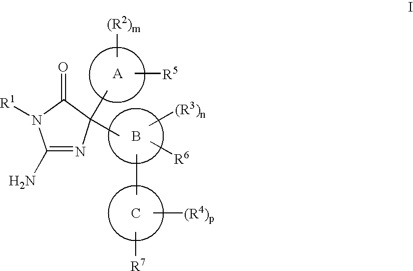
wherein
A is selected from C₁₋₆alkyl, C₂₋₆alkenyl, C₂₋₆alkynyl, C₃₋₆cycloalkyl, C₅₋₇cycloalkenyl, C₆₋₈cycloalkynyl, aryl, heteroaryl, heterocyclyl, C₁₋₆alkylC₃₋₆cycloalkyl, C₁₋₆alkylaryl, C₁₋₆alkylheteroaryl and C₁₋₆alkylheterocyclyl, wherein said C₁₋₆alkyl, C₂₋₆alkenyl, C₂₋₆alkynyl, C₃₋₆cycloalkyl, C₅₋₇cycloalkenyl, C₆₋₈cycloalkynyl, aryl, heteroaryl, heterocyclyl, C₁₋₆alkylC₃₋₆cycloalkyl, C₁₋₆alkylaryl, C₁₋₆alkylheteroaryl or C₁₋₆alkylheterocyclyl is optionally substituted with one or more R⁵;
B is selected from aryl and heteroaryl, wherein said aryl or heteroaryl is optionally substituted with one or more R⁶;
C is selected from aryl, heterocyclyl and heteroaryl, wherein said aryl, heterocyclyl or heteroaryl is optionally substituted with one or more R⁷;
R¹ is selected from hydrogen, C₁₋₆alkyl, C₂₋₆alkenyl, C₂₋₆alkynyl, C₃₋₆cycloalkyl, C₅₋₇cycloalkenyl, C₆₋₈cycloalkynyl, aryl, heteroaryl, heterocyclyl, C₁₋₆alkylC₃₋₆cycloalkyl, C₁₋₆alkylaryl, C₁₋₆alkylheteroaryl and C₁₋₆alkylheterocyclyl, wherein said C₁₋₆alkyl, C₂₋₆alkenyl, C₂₋₆alkynyl, C₃₋₆cycloalkyl, C₅₋₇cycloalkenyl, C₆₋₈cycloalkynyl, aryl, heteroaryl, heterocyclyl, C₁₋₆alkylC₃₋₆cycloalkyl, C₁₋₆alkylaryl, C₁₋₆alkylheteroaryl or C₁₋₆alkylheterocyclyl is optionally substituted with one, two or three D;
R², R³ and R⁴ is Si(R⁸)₃;
R⁵, R⁶ and R⁷ is independently selected from halogen, nitro, CHO, C₀₋₆alkylCN, OC₁₋₆alkylCN, C₀₋₆alkylOR⁹, OC₂₋₆alkylOR⁹, C₀₋₆alkylNR⁹R¹⁰, OC₂₋₆alkylNR⁹R¹⁰, OC₂₋₆alkylOC₂₋₆alkylNR⁹R¹⁰, NR⁹OR¹⁰, C₀₋₆alkylCO₂R⁹, OC₁₋₆alkylCO₂R⁹, C₀₋₆alkylCONR⁹R¹⁰, OC₁₋₆alkylCONR⁹R¹⁰, OC₂₋₆alkylNR⁹(CO)R¹⁰, C₀₋₆alkylNR⁹ (CO)R¹⁰, O(CO)NR⁹R¹⁰, NR⁹(CO)OR¹⁰, NR⁹(CO)NR⁹R¹⁰, O(CO)OR⁹, O(CO)R⁹, C₀₋₆alkylCOR⁹, OC₁₋₆alkylCOR⁹, NR⁹(CO)(CO)R⁹, NR⁹(CO)(CO)NR⁹R¹⁰, C₀₋₆alkylSR⁹, C₀₋₆alkyl(SO₂)NR⁹R¹⁰, OC₁₋₆alkylNR⁹(SO₂)R¹¹, OC₀₋₆alkyl(SO₂)NR⁹R¹⁰, C₀₋₆alkyl(SO)NR⁹R¹⁰, OC₁₋₆alkyl(SO)NR⁹R¹⁰, OSO₂R⁹, SO₃R⁹, C₀₋₆alkylNR⁹(SO₂)NR⁹R¹⁰, C₀₋₆alkylNR⁹(SO)R¹¹, OC₂₋₆alkylNR⁹(SO)R⁹, OC₁₋₆alkylSO₂R⁹, C₁₋₆alkylSO₂R⁹, C₀₋₆alkylSOR⁹, C₁₋₆alkyl, C₂₋₆alkenyl, C₂₋₆alkynyl, C₀₋₆alkylC₃₋₆cycloalkyl, C₀₋₆alkylC₅₋₇cycloalkenyl, C₀₋₆alkylC₆₋₈cycloalkynyl, C₀₋₆alkylaryl, C₀₋₆alkylheteroaryl, C₀₋₆alkylheterocyclyl, and OC₂₋₆alkylheterocyclyl, wherein said C₁₋₆alkyl, C₂₋₆alkenyl, C₂₋₆alkynyl, C₀₋₆alkylC₃₋₆cycloalkyl, C₀₋₆alkylC₅₋₇cycloalkenyl, C₀₋₆alkylC₆₋₈cycloalkynyl, C₀₋₆alkylaryl, C₀₋₆alkylheteroaryl, C₀₋₆alkylheterocyclyl or OC₂₋₆alkylheterocyclyl is optionally substituted by one or more D, and wherein the individual aryl or heteroaryl groups of C₀₋₆alkylaryl or C₀₋₆alkylheteroaryl is optionally fused with a 4, 5, 6 or 7 membered cycloalkyl, cycloalkenyl or heterocyclyl group to form a bicyclic ring system where the bicyclic ring system is optionally substituted with from one or more D;
R⁸ is selected from hydrogen, C₁₋₆alkyl, C₂₋₆alkenyl, C₂₋₆alkynyl, C₀₋₆alkylOR¹¹, C₀₋₆alkylC₃₋₆cycloalkyl, C₀₋₆alkylC₅₋₇cycloalkenyl, C₀₋₆alkylC₆₋₈cycloalkynyl, C₀₋₆alkylaryl, C₀₋₆alkylheteroaryl, C₀₋₆alkylheterocyclyl and C₀₋₆alkylNR¹¹R¹², wherein said C₁₋₆alkyl, C₂₋₆alkenyl, C₂₋₆alkynyl, C₀₋₆alkylC₃₋₆cycloalkyl, C₀₋₆alkylC₅₋₇cycloalkenyl, C₀₋₆alkylC₆₋₈cycloalkynyl, C₀₋₆alkylaryl, C₀₋₆alkylheteroaryl or C₀₋₆alkylheterocyclyl is optionally substituted with one or more D;
R⁹ and R¹⁰ are independently selected from hydrogen, halogen, C₁₋₆alkyl, C₂₋₆alkenyl, C₂₋₆alkynyl, C₀₋₆alkylC₃₋₆cycloalkyl, C₀₋₆alkylC₅₋₇cycloalkenyl, C₀₋₆alkylC₆₋₈cycloalkynyl, C₂₋₆alkenylC₃₋₆cycloalkyl, C₂₋₆alkenylC₅₋₇cycloalkenyl, C₂₋₆alkenylC₆₋₈cycloalkynyl, C₀₋₆alkylaryl, C₀₋₆alkylheteroaryl, C₀₋₆alkylheterocyclyl, C₀₋₆alkylOR¹¹, C₀₋₆alkylNR¹¹R¹², aryl, and heteroaryl, wherein said C₁₋₆alkyl, C₂₋₆alkenyl, C₂₋₆alkynyl, C₀₋₆alkylC₃₋₆cycloalkyl, C₀₋₆alkylC₅₋₇cycloalkenyl, C₀₋₆alkylC₆₋₈cycloalkynyl, C₀₋₆alkylaryl, C₀₋₆alkylheteroaryl, C₀₋₆alkylheterocyclyl, aryl or heteroaryl is optionally substituted by one or more D; or
R⁹ and R¹⁰ may together form a 4 to 6 membered heterocyclic ring containing one or more heteroatoms selected from N, O or S that is optionally substituted with one or more D;
whenever two R⁹ groups occur in the structure they may optionally together form a 5 or 6 membered heterocyclic ring containing one or more heteroatoms selected from N, O or S, that is optionally substituted with one or more D;
R¹¹ and R¹² are independently selected from hydrogen, halogen, C₁₋₆alkyl, C₂₋₆alkenyl, C₂₋₆alkynyl, C₀₋₆alkylC₃₋₆cycloalkyl, C₀₋₆alkylC₅₋₇cycloalkenyl, C₀₋₆alkylC₆₋₈cycloalkynyl, C₀₋₆alkylaryl, C₀₋₆alkylheterocyclyl and C₀₋₆alkylheteroaryl, wherein said C₁₋₆alkyl, C₂₋₆alkenyl, C₂₋₆alkynyl, C₀₋₆alkylC₃₋₆cycloalkyl, C₀₋₆alkylC₅₋₇cycloalkenyl, C₀₋₆alkylC₆₋₈cycloalkynyl, C₀₋₆alkylaryl, C₀₋₆alkylheteroaryl or C₀₋₆alkylheterocyclyl is optionally substituted with one or more D; or
R¹¹ and R¹² may together form a 4 to 6 membered heterocyclic ring containing one or more heteroatoms selected from N, O or S optionally substituted with one or more D;
D is selected from halogen, nitro, COOH, CN, OR¹³, C₁₋₆alkyl, C₂₋₆alkenyl, C₂₋₆alkynyl, C₀₋₆alkylaryl, C₀₋₆alkylheteroaryl, C₀₋₆alkylC₃₋₆cycloalkyl, C₀₋₆alkylC₅₋₇cycloalkenyl, C₀₋₆alkylC₆₋₈cycloalkynyl, C₀₋₆alkylheterocyclyl, OC₂₋₆alkylNR¹³R¹⁴, NR¹³R¹⁴, CONR¹³R¹⁴, NR¹³(CO)R¹⁴, O(CO)R¹³ (CO)OR¹³, COR¹³, (SO₂)NR¹³R¹⁴, NSO₂R¹³, SO₂R¹³, SOR¹³, (CO)C₁₋₆alkylNR¹³R¹⁴, (SO₂)C₁₋₆alkylNR¹³R¹⁴, OSO₂R¹³ and SO₃R¹³, wherein said C₁₋₆alkyl, C₂₋₆alkenyl, C₂₋₆alkynyl, C₀₋₆alkylaryl, C₀₋₆alkylheteroaryl, C₀₋₆alkylheterocyclyl, C₀₋₆alkylC₃₋₆cycloalkyl C₀₋₆alkylC₅₋₇cycloalkenyl or C₀₋₆alkylC₆₋₈cycloalkynyl is optionally substituted with halogen, OSO₂R¹³, SO₃R¹³, nitro, CN, OR¹³, C₁₋₆alkyl;
R¹³ and R¹⁴ are independently selected from hydrogen, halogen, C₁₋₆alkyl, C₃₋₆cycloalkyl, aryl, heteroaryl or heterocyclyl wherein said C₁₋₆alkyl, C₃₋₆cycloalkyl, aryl, heteroaryl or heterocyclyl is optionally substituted with one, two or three hydroxy, CN, halo or C₁₋₃alkyloxy; or
R¹³ and R¹⁴ may together form a 4 to 6 membered heterocyclic ring containing one or more heteroatoms selected from N, O or S optionally substituted with hydroxy, C₁₋₃alkyloxy, cyano or halo;
m=0, 1, 2 or 3;
n=0, 1, 2 or 3;
p=0, 1, 2 or 3;
wherein one of m, n or p is at least 1;
as a free base or a pharmaceutically acceptable salt, solvate or solvate of a salt thereof.

2. A compound according to claim 1, wherein A is aryl.

3. A compound according to claim 1, wherein A is phenyl.

4. A compound according to claim 1, wherein B is aryl.

5. A compound according to claim 1, wherein B is phenyl.

6. A compound according to claim 1, wherein C is aryl, substituted with one or more R⁷.

7. A compound according to claim 6, wherein C is phenyl substituted with one R⁷ and R⁷ represents C₀₋₆alkylOR⁹ and C₀₋₆alkylOR⁹ represents methoxy.

8. A compound according to claim 1, wherein C is hetoraryl.

9. A compound according to claim 1, wherein C is pyrimidine.

10. A compound according to claim 1, wherein C is pyrimidine, substituted with one R⁷ and R⁷ represents methyl or fluoro.

11. A compound according to claim 1, wherein C is pyrazine.

12. A compound according to claim 1, wherein C is pyrazole, substituted with one R⁷ and R⁷ represents methyl.

13. A compound according to claim 1, wherein C is heteroaryl, substituted with one or more R⁷.

14. A compound according to claim 1, wherein C is pyridine, substituted with one R⁷, said R⁷ being halo.

15. A compound according to claim 1, wherein R⁷ represents fluoro or chloro.

16. A compound according to claim 1, wherein C is pyridine, substituted with one R⁷, said R⁷ being C₀₋₆alkylOR⁹ and C₀₋₆alkylOR⁹ represents methoxy.

17. A compound according to claim 1, wherein R¹ is C₁₋₆alkyl.

18. A compound according to claim 1, wherein R¹ is methyl.

19. A compound according to claim 1, wherein R⁸ is C₁₋₆alkyl.

20. A compound according to claim 1, wherein R⁸ is methyl.

21. A compound according to claim 1, wherein m is 1; n is 0; and p is 0.

22. A compound according to claim 1, wherein A is aryl; B is aryl; C is aryl or heteroaryl optionally substituted with one or more R⁷; R⁷ is halo or C₀₋₆alkylOR⁹; R⁹ is C₁₋₆alkyl; R¹ is C₁₋₆alkyl; R⁸ is C₁₋₆alkyl; and m is 1; n is 0; and p is 0.

23. A compound according to claim 1, wherein A is phenyl; B is phenyl; C is phenyl, pyridine or pyrimidine optionally substituted with one or more R⁷; R⁹ is methyl; R¹ is methyl; and R⁸ is methyl.

24. A compound according to claim 1, wherein A is phenyl; B is phenyl; C is phenyl, pyridine, pyrimidine, pyrazine or pyrazole, said phenyl, pyridine, pyrimidine, pyrazine or pyrazole being optionally substituted with one or more R⁷; R⁹ is methyl; R¹ is methyl; and R⁸ is methyl.

25. A compound according to claim 1, selected from:
2-Amino-5-(3′-methoxybiphenyl-3-yl)-3-methyl-5-[4-(trimethylsilyl)phenyl]-3,5-dihydro-4H-imidazol-4-one;
2-Amino-5-[3-(2-fluoropyridin-3-yl)phenyl]-3-methyl-5-[4-(trimethylsilyl)phenyl]-3,5-dihydro-4H-imidazol-4-one;
2-Amino-3-methyl-5-(3-pyrimidin-5-ylphenyl)-5-[4-(trimethylsilyl)phenyl]-3,5-dihydro-4H-imidazol-4-one;
2-amino-5-[3-(5-methoxypyridin-3-yl)phenyl]-3-methyl-5-(4-trimethylsilylphenyl)imidazol-4-one acetic acid salt;
2-amino-3-methyl-5-(3-pyridin-3-ylphenyl)-5-(4-trimethylsilylphenyl)imidazol-4-one acetic acid salt;
2-amino-5-[3-(5-fluoropyridin-3-yl)phenyl]-3-methyl-5-(4-trimethylsilylphenyl)imidazol-4-one acetic acid salt;
5-[3-[2-amino-1-methyl-5-oxo-4-(4-trimethylsilylphenyl)imidazol-4-yl]phenyl]pyridine-3-carbonitrile acetic acid salt;
2-amino-5-[3-(6-fluoropyridin-3-yl)phenyl]-3-methyl-5-(4-trimethylsilylphenyl)imidazol-4-one acetic acid salt;
3-[3-[2-amino-1-methyl-5-oxo-4-(4-trimethylsilylphenyl)imidazol-4-yl]phenyl]pyridine-4-carbonitrile acetic acid salt;
2-amino-3-methyl-5-[3-(1-methylpyrazol-4-yl)phenyl]-5-(4-trimethylsilylphenyl)imidazol-4-one acetic acid salt;
2-amino-3-methyl-5-[3-(2-methylpyrimidin-5-yl)phenyl]-5-(4-trimethylsilylphenyl)imidazol-4-one acetic acid salt;
2-amino-3-methyl-5-(3-pyrazin-2-ylphenyl)-5-(4-trimethylsilylphenyl)imidazol-4-one;
2-amino-5-[3-(2-fluoropyrimidin-5-yl)phenyl]-3-methyl-5-(4-trimethylsilylphenyl)imidazol-4-one;
2-amino-1-methyl-4-(3-(pyrimidin-5-yl)phenyl)-4-(3-(trimethylsilyl)phenyl)-1H-imidazol-5(4H)-one;
2-amino-4-(3-(5-chloropyridin-3-yl)phenyl)-1-methyl-4-(3-(trimethylsilyl)phenyl)-1H-imidazol-5(4H)-one;
2-amino-4-(3-(6-fluoropyridin-3-yl)phenyl)-1-methyl-4-(3-(trimethylsilyl)phenyl)-1H-imidazol-5(4H)-one;
2-amino-1-methyl-4-(3-(pyridin-3-yl)phenyl)-4-(3-(trimethylsilyl)phenyl)-1H-imidazol-5(4H)-one;
2-amino-4-(3-(2-fluoropyridin-3-yl)phenyl)-1-methyl-4-(3-(trimethylsilyl)phenyl)-1H-imidazol-5(4H)-one;
2-amino-4-(3-(6-methoxypyridin-2-yl)phenyl)-1-methyl-4-(3-(trimethylsilyl)phenyl)-1H-imidazol-5(4H)-one; and
2-amino-1-methyl-4-(3-(pyrazin-2-yl)phenyl)-4-(3-(trimethylsilyl)phenyl)-1H-imidazol-5(4H)-one;
as a free base or a pharmaceutically acceptable salt, solvate or solvate of a salt thereof.

26. A pharmaceutical composition comprising as active ingredient a therapeutically effective amount of a compound according to claim 1 in association with a pharmaceutically acceptable excipient, carrier or diluent.

27. A method of inhibiting activity of BACE comprising contacting said BACE with a compound according to claim 1.

28. A method of treating or preventing an Aβ-related pathology in a mammal, comprising administering to said mammal a therapeutically effective amount of a compound according to claim 1.

29. The method of claim 28, wherein said Aβ-related pathology is Downs syndrome, a amyloid angiopathy, cerebral amyloid angiopathy, hereditary cerebral hemorrhage, a disorder associated with cognitive impairment, MCI (“mild cognitive impairment”), Alzheimer Disease, memory loss, attention deficit symptoms associated with Alzheimer disease, neurodegeneration associated with Alzheimer disease, dementia of mixed vascular origin, dementia of degenerative origin, pre-senile dementia, senile dementia, dementia associated with Parkinson's disease, progressive supranuclear palsy or cortical basal degeneration.

30. A method of treating or preventing Alzheimer's Disease in a patient, comprising administering to said patient a therapeutically effective amount of a compound according to claim 1.

31. The method of claim 29, wherein said mammal is a human.

32. The method of claim 30, wherein said patient is a human.

33. A method of treating or preventing an Aβ-related pathology in a mammal, comprising administering to said mammal a therapeutically effective amount of a compound according to claim 1 and at least one cognitive enhancing agent, memory enhancing agent, or choline esterase inhibitor.

34. The method of claim 33, wherein said Aβ-related pathology is Downs syndrome, a amyloid angiopathy, cerebral amyloid angiopathy, hereditary cerebral hemorrhage, a disorder associated with cognitive impairment, MCI (“mild cognitive impairment”), Alzheimer Disease, memory loss, attention deficit symptoms associated with Alzheimer disease, neurodegeneration associated with Alzheimer disease, dementia of mixed vascular origin, dementia of degenerative origin, pre-senile dementia, senile dementia, dementia associated with Parkinson's disease, progressive supranuclear palsy or cortical basal degeneration.

35. The method of claim 34, wherein said Aβ-related pathology is Alzheimer Disease.

36. The method of claim 34, wherein said mammal is a human.
